(19) United States
(12) Patent Application Publication
(10) Pub. No.: US 2025/0253023 A1
(43) Pub. Date: Aug. 7, 2025

(54) Systems and Methods for Compounding Pharmacy

(71) Applicant: Chewy, Inc., Dania Beach, FL (US)

(72) Inventors: Brian Bricker, Medfield, MA (US); Cecilia Cotterman, Issaquah, WA (US); Devyani Totre, Plymouth, MN (US); Esau Cuevas, Conroe, TX (US); Liam Muller, Milford, NH (US); Michael Arrabito, Staten Island, NY (US); Michael Wright, Louisville, KY (US); Patrick Joseph Newell, Andover, MA (US); Poornima Chinnaraj, Carver, MN (US); Ramzi Saba, Arlington, MA (US); Siddharth Atul Parasnis, Somerville, MA (US); Suraj Nair, Medford, MA (US); Swagata Chakroborty, Baltimore, MD (US); Vasilios Hatziioannou, Danvers, MA (US); Rohith Grama, Boston, MA (US); Zachary Laznick, Boston, MA (US); Lucas Hamelink, Louisville, KY (US); Warren David Finlinson, Simpsonville, KY (US)

(21) Appl. No.: 19/046,034

(22) Filed: Feb. 5, 2025

Related U.S. Application Data
(60) Provisional application No. 63/549,747, filed on Feb. 5, 2024.

(51) Int. Cl. G16H 20/10; G06F 3/0484; G06Q 10/0631; G06Q 10/0875; G06Q 30/0202

(52) U.S. Cl. CPC G16H 20/10; G06F 3/0484; G06Q 10/06315; G06Q 10/0875; G06Q 30/0202

(57) ABSTRACT

A system and method are provided for automating preparation of compound prescription at workstations having a user interface and a quantity input hardware, based on forecasted quantities, ordered quantities and simultaneous usage data. The method may include obtaining orders for compound prescriptions, via web clients, tracking information related to inventories, generating compounding tasks corresponding to the orders by analyzing ingredients necessary, prioritizing and assigning the compounding tasks to workstations, based on forecast demand, available supply, capacity, and the inventories, providing graphical user interfaces for users to process the orders at the workstations. In response to detecting a user of processing an order, the method may also include updating the inventories, updating the compounding tasks, and/or providing an update on the graphical user interfaces regarding a next compounding task performable at the workstation.

### RELATED APPLICATIONS

This application claims priority to U.S. Provisional Patent Application No. 63/549,747, filed Feb. 5, 2024, entitled “Systems and Methods for Compounding Pharmacy,” which is incorporated by reference herein in its entirety.

### TECHNICAL FIELD

The disclosed embodiments relate generally to pharmacy management and more specifically to systems and methods for veterinary compounding pharmacy.

### BACKGROUND

A compounding pharmacy is a state-regulated pharmacy that can make prescription drugs for specific patients. Doctors choose compounded medications for their patients when commercial drugs are either not appropriate or such drugs are not available. In a compounding pharmacy, a medication is prepared to exact specifications for each patient. For animals, prescriptions can be especially difficult. Typically, there is a narrow selection of medications manufactured by pharmaceutical companies for treating animals. Compounded medications for animals may include flavored medications, medications with strict size, strength and dosage requirements, and/or combination drugs.

Conventional compounding pharmacies are not equipped to process a large number of concurrent orders or prescriptions. Some compounding pharmacies require many manual steps, are prone to human errors, and/or can be unreasonably slow. Compounding pharmacies also need to comply with Food and Drug Administration (FDA) regulations and may have recalls. Compliance, changes in demand for medications and supply shortages also complicate compounding pharmacies. A pharmacy is managed by a dedicated team of pharmacists and technicians, who are tasked with fulfilling both immediate and projected demands stemming from customer orders. Pharmacists need to efficiently manage technicians, workstations and workflow, and/or control quality for the compounded drugs.

### SUMMARY

Accordingly, there is a need for systems and methods that automate compounding pharmacies to address at least some of the problems described above. Aspects of the system described herein can enable veterinary pharmacists to plan, prioritize and assign compounding tasks to workstations and/or technicians based on customer and forecast demand (e.g., customer orders and forecasted units), available supply (e.g., inventory), and/or capacity (e.g., labor and physical resources). The system may recommend a work plan based on planned shifts of labor and workstations. The work plan may allow pharmacists to make final adjustments as needed. After the plan is finalized, technicians may use a compounding operations dashboard to work on their own task queue quickly and efficiently. The pharmacists may use the dashboard to monitor work-in-progress in real-time, identify any bottlenecks in a timely manner and reassign tasks as needed.

In accordance with some embodiments, a method executes at a computer system having one or more processors and memory storing one or more programs configured for execution by the one or more processors. The system may be used for automating preparation of compound prescription at a plurality of workstations having integrated therein a user interface and a registered quantity input hardware, based on forecasted quantities, ordered quantities and simultaneous usage data. The method may include obtaining a plurality of orders for compound prescriptions, via one or more web clients. The method may also include tracking information related to inventories including expiration dates for different lots of the inventories. The method may also include generating compounding tasks corresponding to the plurality of orders by analyzing ingredients necessary for fulfilling each order and assigning, for each order, a respective one or more lots for an inventory. The method may also include prioritizing and assigning the compounding tasks to a plurality of workstations, based on forecast demand, available supply, capacity, and/or the information related to the inventories. The method may also include providing one or more graphical user interfaces for a plurality of users, to process the plurality of orders at the plurality of workstations. The method may also include in response to detecting a user of the plurality of users processing an order of the plurality of orders at a workstation of the plurality of workstations, updating the information related to the inventories, updating the compounding tasks, and/or providing an update on the one or more graphical user interfaces regarding a next compounding task performable at the workstation.

In some embodiments, the method may include interfacing with at least one device selected from the group consisting of: one or more scales, one or more scanners, and one or more mixers, and detecting a status of the order of the plurality of orders at the workstation, by obtaining one or more signals from the at least one device.

In some embodiments, the method may further include retrieving one or more templates corresponding to the plurality of orders, wherein each template includes a set of instructions for a workflow and a formula, and/or providing, via the one or more graphical user interfaces, details to implement the formula using the workflow, at the plurality of workstations.

In some embodiments, the one or more templates are customizable based upon one or more devices connected to each workstation.

In some embodiments, the method may include providing to a first set of users of the plurality of users a dashboard to update the one or more templates, and/or upon receiving an update the one or more templates, providing the updates, via the one or more graphical user interfaces.

In some embodiments, the capacity may correspond to at least one of a number of workstations, and/or a number of users.

In some embodiments, the available supply may be tracked based on tracking information related to the inventories, at one or more warehouses, and/or at the plurality of workstations.

In some embodiments, the forecast demand may be obtained based on a forecasting model that is trained to predict demand based on historical data for compound prescription from the one or more web clients.

In some embodiments, prioritizing and assigning the compounding tasks to the plurality of workstations may be based on planned shifts of the plurality of users, and/or operational efficiency of the plurality of workstations.

In some embodiments, prioritizing and assigning the compounding tasks to the plurality of workstations may be based on forecasting, inventory, outstanding orders, a time an order was approved, and/or target days of coverage.

In some embodiments, the method may further include providing, via the one or more graphical user interfaces, for the user, while the user is processing the order, a real-time update regarding a weight of an ingredient and an expected range of weight for the ingredient according to a formula. The method may also include in response to detecting the weight of the ingredient is stabilized, displaying a final weight of the ingredient, allowing the user to record, and/or automatically recording the final weight of the ingredient for the order.

In some embodiments, the method may further include providing, via the one or more graphical user interfaces, for a super user different from the user processing the order, after the user processes the order, a summary of the processing of the order. The method may also include in response to receiving a negative input from the super user regarding an incorrect compounding for the order, requeuing a task corresponding to the order to the plurality of workstations.

In some embodiments, prioritizing and assigning the compounding tasks to the plurality of workstations may be based on batching one or more tasks that use an ingredient.

In some embodiments, prioritizing and assigning the compounding tasks to the plurality of workstations may be based on ordering tasks for one or more customers responsible for the plurality of orders received via the one or more web clients.

In some embodiments, prioritizing and assigning the compounding tasks to the plurality of workstations may be based on a demand for one or more ingredients.

In some embodiments, the method may include prioritizing and assigning the compounding tasks to the plurality of workstations may be based on task initiation prompted by a user request through the one or more graphical user interfaces thereby providing on-demand control.

In some embodiments, the method may include prioritizing and assigning the compounding tasks to the plurality of workstations may be automatically triggered through a daemon to execute scheduled commands job.

In some embodiments, each compound may include a corresponding minimum inventory threshold to trigger a new order and/or a minimum batch size for a next order.

In some embodiments, the one or more processors may be configured to perform any of the methods described herein.

In accordance with some embodiments, a method executes at a computer system having one or more processors and memory storing one or more programs configured for execution by the one or more processors. The system may be used for guiding the fulfillment of a compound pharmaceutical product. The method may include rendering on a pharmacist user interface, a plurality of fields configured to receive input from a user having a first level authority. The method may also include receiving ingredient data to create an active formulation via a plurality of fields corresponding to i) pharmaceutical ingredients in a formulary; ii) quantities of the pharmaceutical ingredients making up the compound pharmaceutical product; iii) instructions for combining the pharmaceutical ingredients to produce the compound pharmaceutical product. The method may also include receiving a digital order for the compound pharmaceutical product. In response to receiving the digital order, the method may also include rendering on a workstation UI identified by a computer readable indicia, instructions for formulating the compound pharmaceutical product and compounding fields configured to receive input from a user having a second level of authority that is different from the first level of authority. The method may also include digitally recording, based on the computer readable indicia, receipt of pharmaceutical ingredients for formulating the compound pharmaceutical product. The method may also include receiving progressive inputs based on authorization from the user having a second level of authority into the compounding fields, the inputs being associated with quantities of pharmaceutical ingredients that are dispensed proximate the workstation UI. The method may also include receiving a digital completion entry from at the workstation UI indicating the digital order has been created. The method may also include reconciling the quantities of pharmaceutical ingredients that have been dispensed in the creation of the compound pharmaceutical product with indicated quantities in the instructions for formulating the compound pharmaceutical product.

In some embodiments, the receiving progressive inputs may be automated via a digital input device coupled to the workstation UI and wherein the digital input device is taken from the group consisting of a digital scale, a mixing device, and combinations thereof.

In some embodiments, digitally recording the receipt of pharmaceutical ingredients may include reading input from a bar code scanner indicating that the scanner is associated with the workstation UI.

In some embodiments, the method may include indicating in a formulation database that the active formulation supersedes all prior formulations.

The method may also include creating a compound pharmaceutical template via plurality of active formulations.

In some embodiments, rendering on a workstation UI identified by a computer readable indicia, instructions for formulating the compound pharmaceutical product, may be based on a ranking based on one or more factors comprising a time since order was placed, time since prescription was approved, and/or inventory levels.

In another aspect, an electronic device includes one or more processors, memory, a display, and one or more programs stored in the memory. The programs are configured for execution by the one or more processors and are configured to perform any of the methods described herein, according to some embodiments.

In another aspect, a non-transitory computer readable storage medium stores one or more programs configured for execution by a computing device having one or more processors, memory, and a display. The one or more programs are configured to perform any of the methods described herein, according to some embodiments.

Thus, methods and systems are disclosed that automate compounding pharmacies.

Both the foregoing general description and the following detailed description are exemplary and explanatory, and are intended to provide further explanation of the invention as claimed.

### BRIEF DESCRIPTION OF THE DRAWINGS

For a better understanding of the aforementioned systems, methods, and graphical user interfaces, as well as additional systems, methods, and graphical user interfaces that provide data visualization analytics, reference should be made to the Description of Embodiments below, in conjunction with the following drawings in which like reference numerals refer to corresponding parts throughout the figures.

FIG. 1A shows a block diagram of an example system for compounding pharmacy, according to some embodiments.

FIG. 1B shows another example system for compounding pharmacy, according to some embodiments.

FIG. 2 shows a schematic diagram for an example demand processing, according to some embodiments.

FIG. 3 is a schematic diagram of an example system comprising a compounding pharmacy lab and an operations portal hosted in a private cloud, according to some embodiments.

FIG. 4A shows an example label, according to some embodiments.

FIG. 4B shows an example user interface for recording a current weight for an ingredient, according to some embodiments.

FIG. 4C shows an example user interface for rescanning an ingredient, according to some embodiments.

FIG. 4D shows an example user interface for confirming a state of the lab or the interface, according to some embodiments.

FIG. 4E shows an example user interface for filing and/or sample weighing, according to some embodiments.

FIG. 4F shows an example user interface for logging a workstation, according to some embodiments.

FIG. 4G shows an example user interface for recording a yield, according to some embodiments.

FIG. 4H shows an example user interface for calibrating an empty beaker, according to some embodiments.

FIGS. 5A-5E show a flow diagram of an example compounding workflow, according to some embodiments.

FIG. 6A shows a flowchart for an example process for calculating demand, according to some embodiments.

FIG. 6B shows a flowchart for an example process for creating tasks, according to some embodiments.

FIGS. 7A and 7B show a flowchart of an example process for updating formula to lot task cascading, according to some embodiments.

FIG. 8 shows an example user interface for configuring ranking factors, according to some embodiments.

FIG. 9 shows another example user interface for configuring ranking factors, according to some embodiments.

FIG. 10 shows a flow diagram of an example flow for sub-formula task creation, according to some embodiments.

FIG. 11 shows an example user interface for sub-formula task rebalancing, according to some embodiments.

FIG. 12 shows a flow diagram of an example process for sub-formula task rebalancing, according to some embodiments.

FIG. 13A shows an example template landing page, according to some embodiments.

FIG. 13B shows an example template creation workflow when a pharmacist initiates a new version, according to some embodiments.

FIG. 13C shows an example user interface for viewing and/or updating an existing template, according to some embodiments.

FIG. 13D shows an example formula management landing page, according to some embodiments.

FIG. 13E shows an example user interface for viewing and/or updating an existing formula, according to some embodiments.

FIG. 13F shows an example user interface for creating a newer version from an existing formula, according to some embodiments.

FIG. 13G shows an example user interface for viewing version history, according to some embodiments.

FIG. 13H shows an example user interface for bulk upload landing page, which may be used for performing uploads, according to some embodiments.

FIGS. 14A-14J show example flow diagrams for formula management, according to some embodiments.

FIGS. 15A-15F show example flow diagrams for ingredient management, according to some embodiments.

FIG. 16 is a schematic diagram for example demand and planning, according to some embodiments.

FIG. 17 shows a flow chart of an example method for automating compounding pharmacy, according to some embodiments.

FIG. 18 shows a flow chart of an example method for guiding the fulfillment of a compound pharmaceutical product, according to some embodiments.

Reference will now be made to embodiments, examples of which are illustrated in the accompanying drawings. In the following description, numerous specific details are set forth in order to provide a thorough understanding of the present invention. However, it will be apparent to one of ordinary skill in the art that the present invention may be practiced without requiring one or more of these specific details.

### DESCRIPTION OF EMBODIMENTS

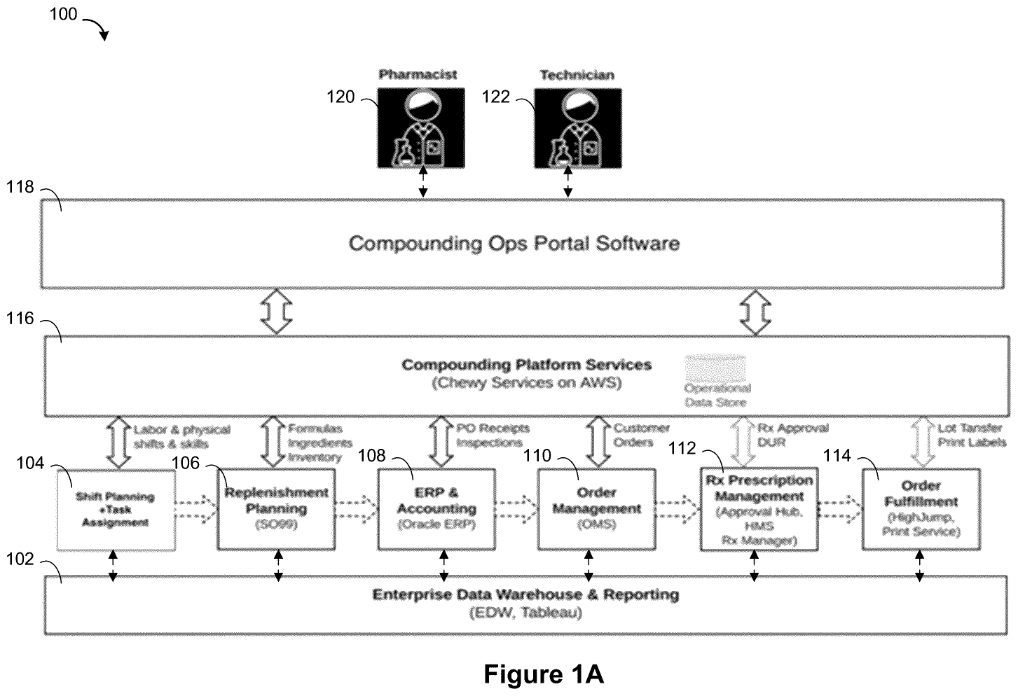

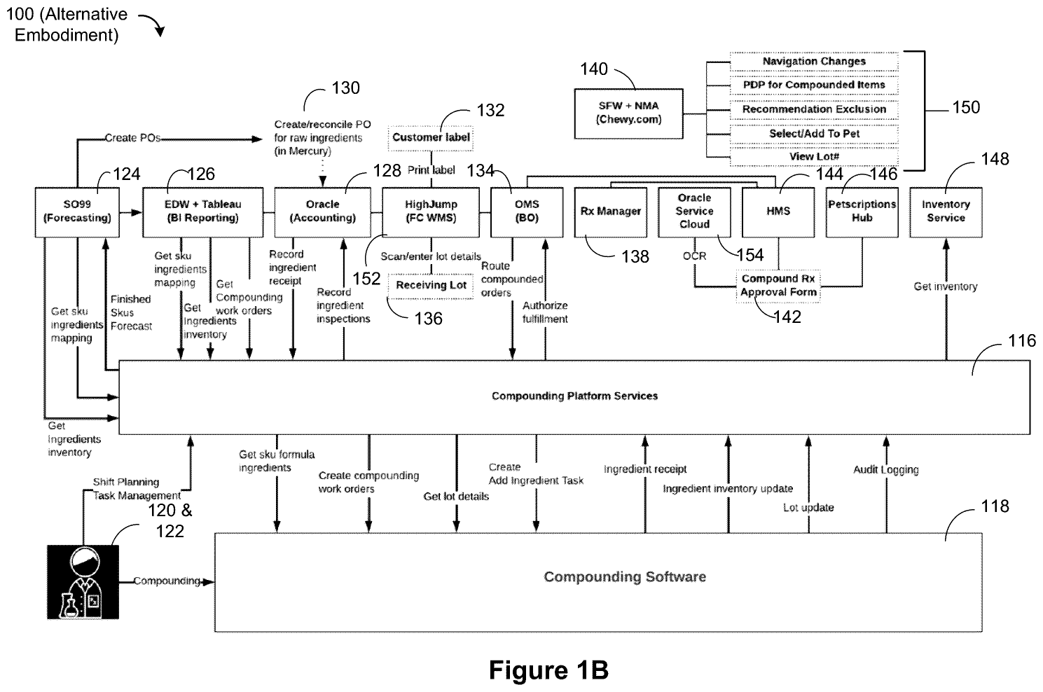

FIG. 1A shows a block diagram of an example system 100 for compounding pharmacy, according to some embodiments. A pharmacy may be managed by a team of pharmacists 120 and/or technicians 122, tasked with fulfilling immediate and/or projected demands stemming from customer orders (e.g., approximately 8,000 SKUs) on a web portal (e.g., a business-to-consumer (B2C) portal), such as chewy.com. A goal of the system may be to provide compounding expansion (e.g., sterile compounding facility, adding new facilities to provide compounding), provide access to a variety of products. Some embodiments extend this service to veterinarians (business-to-business or B2B). Described herein are examples of compounding creation workflows that may include operations for making medications. A high-level description of the different components and functionalities within an example pharmacy framework is provided followed by example details.

Systems, methods, interfaces and techniques described herein can help pharmacists 120 plan, prioritize, and/or assign compounding tasks to technicians 122 based on customer and forecast demand (e.g., customer orders and forecasted SKUs), available supply (e.g., inventory), and/or capacity (e.g., labor and physical resources). The system may recommend a work plan based on planned shifts of labor and workstations, and/or it may allow pharmacists 120 to make final adjustments as needed. After a plan is finalized, technicians 122 may use a compounding operations dashboard (e.g., a dashboard provided via a compounding operations portal software 118) to work on their own task queue quickly and efficiently. The pharmacists 120 may use the dashboard to monitor work-in-progress in real-time, identify any bottlenecks in a timely manner, and/or reassign tasks as needed.

The compounding operations portal software 118 may interface with a compounding platform services, which may be a cloud service hosted on a cloud platform (e.g., Amazon AWS, Microsoft Azure, Google Cloud). The compounding platform services in turn obtains labor and physical shifts and skills from a shift and planning task assignment 104, formulas, ingredients and/or inventory from a replenishment planning 106, purchase order receipts and/or inspections from ERP and accounting 108, customer orders from an order management system (OMS) 110, prescription approval from prescription management 112, and/or lot transfer or print labels from order fulfillment 114.

FIG. 1B shows an embodiment of the example system 100. A pharmacist 120 and/or a technician 122 may interface with the compounding software 118 for compounding tasks. The pharmacist 120 may perform shift planning and/or task management using the compounding platform services 116. The compounding platform services 116 may provide SKU formula ingredients, create compounding work orders, obtain lot details, and/or create and/or add ingredient tasks, for the compounding software 118. The compounding platform services 116 may use ingredient receipt, ingredient inventory update, lot update, and/or audit logging, from the compounding software 118. The compound platform services 116 may obtain SKU ingredient mapping, and/or ingredient inventory, from forecasting 124, and supply finished SKUs and/or forecast to the forecasting 124. An enterprise data warehouse (EDW) is one or more databases that may centralize an organization's information from multiple sources and applications, and makes the information available for analytics and/or other use across the organization. The EDW and data visualization and/or reporting 126 may obtain forecast data from forecasting 124, and/or provide SKU ingredients mapping, ingredients inventory, and/or compounding work orders to the compounding platform services 116. Accounting 128 (sometimes referred to as Oracle) may obtain purchase orders from forecasting 124, and/or create/reconcile purchase order for raw ingredients (130), obtain reports from reporting 126. The compounding platform services 116 may record ingredient inspections using the accounting 128 which may provide a record ingredient receipt.

The accounting 128 may be connected to a fulfillment center warehouse management system (FC WMS) 152, sometimes referred to as High Jump, which may be used to generate customer labels, and/or scan/enter lot details to obtain receiving lots 136, based on information obtained from the accounting 128 and/or an order management service (OMS) 1345. The OMS 134 may use information obtained from the FC WMS 152 to route compounded orders to, and/or receive authorize fulfillment from, the compounding platform services 116. The system 100 may also include a prescription manager 138 which may be connected to a health management service (HMS) 144 for managing and/or providing health services. The system 100 may also include a service 154 for obtaining compound prescription approval form 142 (e.g., scan such a form using optical character recognition). The HMS 144 and/or a prescriptions hub 146 (for providing and/or managing prescriptions) may also use the form 142. The inventory service 148 may obtain inventory from the compounding platform services 116.

In some embodiments, if order items are present and approved in the OMS event, for each order item in the database, if the order item is not in a terminal state, and is no longer approved in the OMS event, the status may be set to new, resolved time may be removed, and/or approval status may be set to pending. This generally may mean that the pharmacist or fulfillment has rejected the order, and another order release approval may be expected.

For order releasing, the OMS 134 may determine if each valid part number (e.g., a SKU number, a six-digit number) has status prescription approved orders and if there is sufficient unallocated quantity to allocate to these orders. Status may be updated to inventory found. The HMS 144 may make sure there are prescription approved orders for each part number, that available quantity data is not stale. If those checks are successful, some embodiments also make sure there is unallocated quantity available to fill these prescription approved orders. For each part number, some embodiments may check orders to release if the count of prescription approved orders is greater than 0 and if the part number is not blank. Some embodiments may check the last date of quantity updated and warn if older than a predetermined time period (e.g., 15 minutes). Or if there were any shipped orders since the last time the quantity was updated, may be shipped order cause some issue. Some embodiments log a warning if one of the above two conditions are true, which probably means formula demand summary for this part number is stale. In that case, the system may also retrieve available quantity from the inventory service and/or overwrite the current formula demand summary's available quantity variable. After these checks, the system may calculate unallocated quantity (total available quantity minus inventory found quantity). If there is stale data and based on retrieval of the available quantity from the inventory service, this part number may not be eligible for the release of any orders. Or if this unallocated quantity is nonpositive, it may mean there is no inventory for this part number available to release and this part number may be ineligible to release any of its prescription approved orders. Some embodiments log a warning if there is no inventory. Inventory service 148 may provide real-time information on availability of compounds. After finding the part numbers which have an available unallocated quantity that can be assigned to approved orders, some embodiments start releasing orders. In some embodiments, all orders are sorted for a particular part number into their statuses (e.g., prescription approved, inventory found). If the list of approved orders is not empty or null (meaning this part number has prescription approved numbers looking for inventory). Some embodiments calculate unallocated available quantity using the formula total available quantity minus inventory found order quantities summed up. Some embodiments take unallocated available quantity and try to release as many prescription approved orders it can with that quantity (releasing may update the status from prescription approved to inventory found). Some embodiments releases orders on a first-come-first served bases. Some embodiments send a notification on releasing an order. After an order is successfully released, the total unallocated available quantity may be recalculated.

Order item synchronization (e.g., a cron job) may be run as a part of a handle forecast lambda. Some embodiments also synchronize orders before processing OMS events. Order item synchronization may look up order item that have not been updated within the past few days (e.g., 2 days) having a status non pharmacy blocked, new, prescription approved, inventory found. Some embodiments call order service for such orders and/or update the following details. Synchronization status may update status of order item based on order service response (e.g., order status is shipped or deposited, any items on event does not match a legacy identifier). If the determined status is different from order status, status may be updated with data service. Order notification service's synchronize order may process order notifications from OMS containing header contains pharma; this may act as the first filter towards processing an OMS event. For the following scenarios, the OMS event may be ignored: compare the OMS order identifier and OMS time emitted from an order item table is newer compared to the webhook (a newer event was probably processed before this); order created, edited, item quantity adjusted. Some embodiments synchronize the order before processing these events. Some embodiments also ignore order items that are non-pharma and/or not compounded based on headers received from an OMS event. Some embodiments synchronize creates, updates and/or cancels order items based on received webhook. Some embodiments synchronize order service using Spring Boot with GraphQL using ApolloClient. Some embodiments use part number order identifier to query order service. Order synchronization may work on order statuses. Order status may be marked as shipped within data service if order status is in the following statutes: shipped, deposited, and/or fulfillment status shipped. Order status may be marked cancelled if OMS order status is cancelled and/or item identifier is missing from OMS order. The query may time out in predetermined time (e.g., 10 seconds) with an error retrieving data for order. The order service synchronization may be run when an event is received from OMS, and/or at a predetermined time (e.g., 5 AM, before loading in forecast file handler).

Inventory synchronization (e.g., a cron job in a platform service) may be run at a predetermined interval (e.g., every 5 minutes) to synchronize prescription item inventory table with inventory quantities from inventory service. In some embodiments, a prescription database stores inventory for a given part in an item inventory table. This information may be used to determine if lot tasks need to be created (lot task demand processing), and/or order can be released (order releasing). A part number's inventory may be fetched if there are any outstanding orders or forecasts. Lot task ranking and/or demand processing may use the forecast. A forecast table may have weekly forecasts by part and area (sometime referred to as a warehouse). Import process may include obtaining formula identifiers (e.g., from a formula identifier table), obtaining Oracle (accounting) identifiers, for every forecast from the S3 file. Some embodiments ignore if the identifier is not a compound identifier. If there is an existing forecast for that part and week, some embodiments update a current row, otherwise a new row may be created.

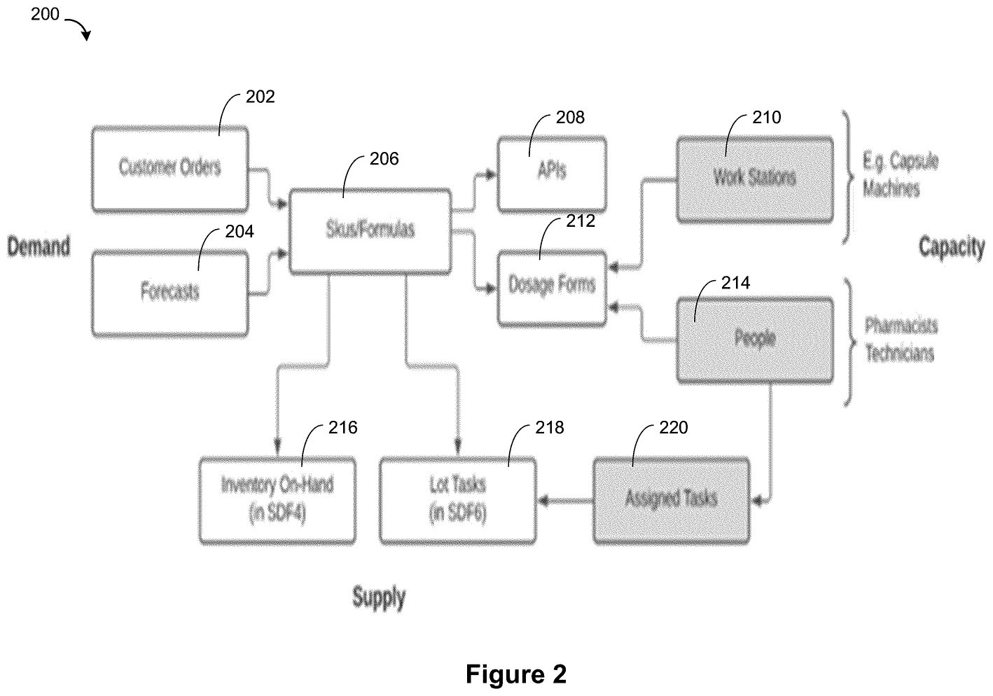

FIG. 2 shows a schematic diagram for an example demand processing 200, according to some embodiments. Customer orders 202 and forecasts 202 may be used to obtain SKUs and/or formulas 206. The SKUs and/or formulas 206 may be used to obtain inventory on-hand 216, lot tasks 218, and/or dosage forms 212, and/or respond to application programming interfaces (APIs) 208. The dosage forms 212 may be further derived from or based on input from workstations 210 and/or input from pharmacists and/or technicians 214. The pharmacists and/or technicians 214 may provide or assign tasks 220 which may be used to obtain the lot tasks 218 based on the SKUs/formulas 206.

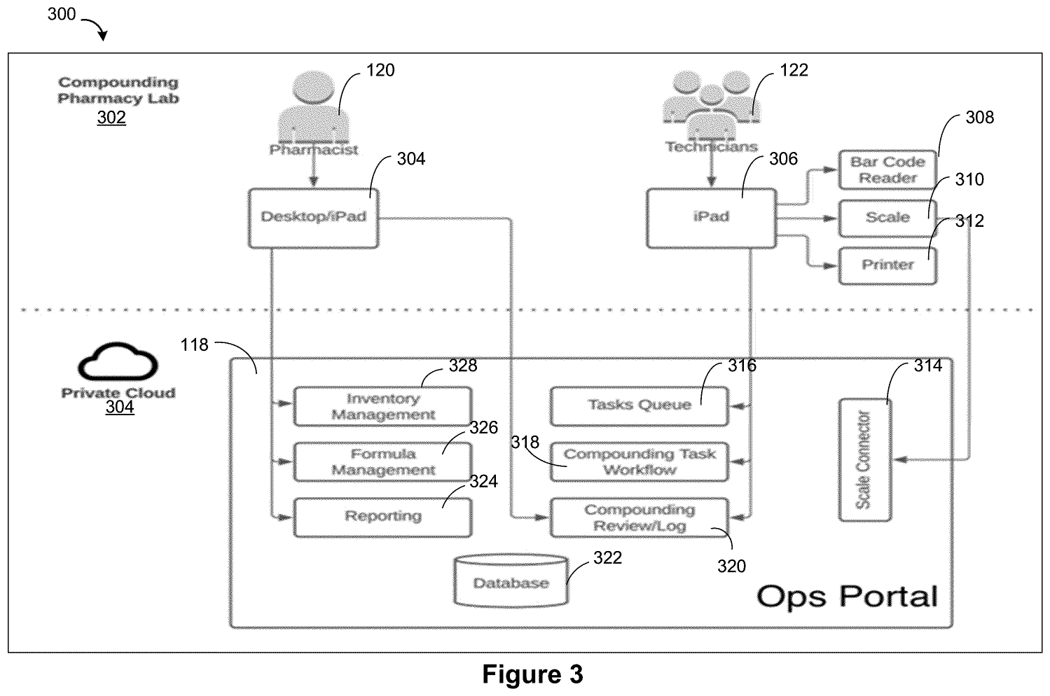

FIG. 3 is a schematic diagram of an example system 300 comprising a compounding pharmacy lab 302 and an operations portal hosted in a private cloud 304, according to some embodiments. The operations portal is sometimes referred to as an OPS portal (e.g., user interfaces for compounding). The compounding ops portal software 118 is an example of the ops portal. The ops portal 118 may include inventory management 328, formula management 326, reporting system 324, tasks queue 316, compounding task workflow 318, compounding review or log 320, and/or scale connector 314. The inventory management system 328 may define and track inventory of ingredients and formulas, and/or receive and inspect ingredients. The operational manager or a pharmacist 120 may use the inventory management system 328 via a computing device 304 (e.g., a desktop or an iPad). The pharmacist 120 may also use the formula management system 326 (via the computing device 304 or another device) to define formulas and/or ingredient ratios and formulation tasks. The pharmacist 120 may also use the reporting system 324 (via the computing device 304 or another device) that provides basic reporting including inventory and tasks. The tasks queue management system 316 may provide basic queue of tasks including lot creation and review tasks, formula reviews, and/or provide an ability to filter by priority (e.g., three priority levels), due date, and/or search (e.g., search by formula name). The technician 122 and/or the pharmacist 120 may use the tasks queue 316.

The compounding task workflow 318 may include start of a compounding task. A first user who begins work on a task may become the default owner of the task. The compound task workflow (sometimes referred to as a compounding workflow) may include performance of formula tasks. This may include reviewing documentation, cleaning, taking photos, mixing, changing gloves, measuring, refrigeration, and/or packaging. The compounding workflow may also include measuring and recording ingredients, signing off on measurements, completing a compounding task, and/or reviewing and approving a compounding task, via the compounding review of log 320. The technician 122 and/or the pharmacist 120 may perform steps of the compounding workflow 318.

Steps and sub-steps in the compounding workflow 318 may be built using formula and/or tasks. Compounding recipes may be separated into steps, and sub-steps. Compounding recipes may be referred to as a medication formula or a formula. A lot task may outline the sequential steps for compounding a medication, incorporating both the formula procedure, ingredient measurements and/or required equipment(s). Every lot task may have a respective formula. A formula may hold the configuration options for lot tasks assigned to that formula. This may include a type of a lot task, any flags, and/or any ingredients that may be required. A template indicates what a recipe may look like. A template may be used in multiple formula. Task objects may hold values relevant to a lot task implementation of that step or sub-step, such as who completed the sub-step, who approved the step, and what equipment was scanned. Some embodiments have an endpoint that returns this information to drive a workflow display. Steps may be containers that hold sub-steps.

A formula may be made of multiple list steps (e.g., a recipe), single weight step, and/or a single final approval step. Sub-steps may be individual components of a business logic that drives the compounding workflow. Each sub-step may be tailored to a specific piece of the compounding workflow. Each sub-step may have their own requirements and logic for when they are considered complete. Step properties may include a property that presents a “Begin” button that must be clicked to start the step. Step properties may also include a property that requires that a compounder requests approval and that a pharmacist that is not a step compounder approves the step. Step properties may also include a property that provides a notes section at the end of the step that can be edited by any user. Step properties may also include instructions that are additional text that can be provided to display to the compounder. Step properties may also include a property that indicates whether the compounder should be allowed to upload photos for the step, if it is required to upload photos for the step to be complete, or to not show any photo option.

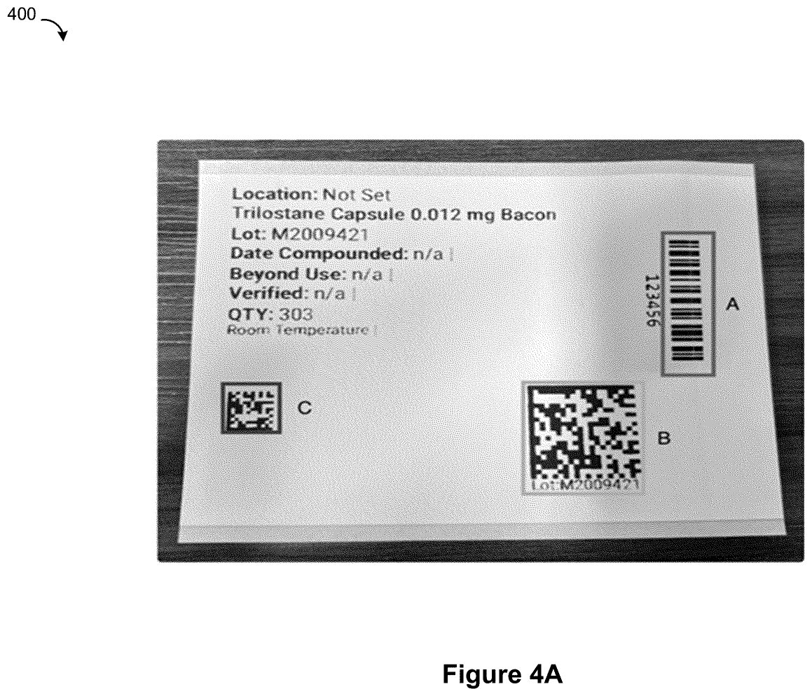

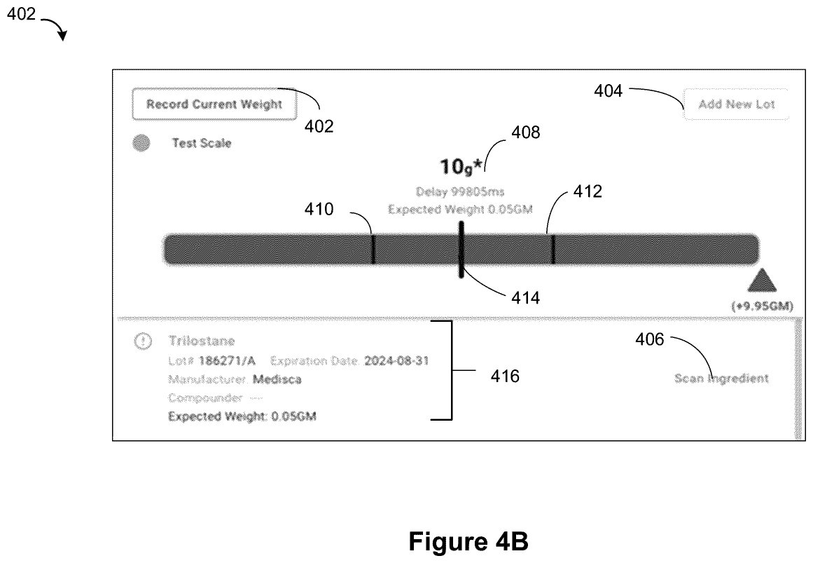

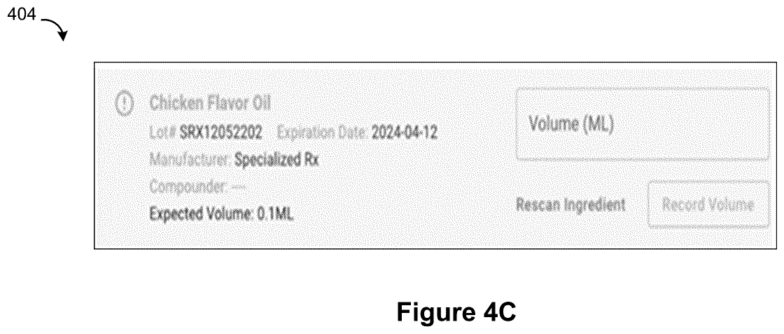

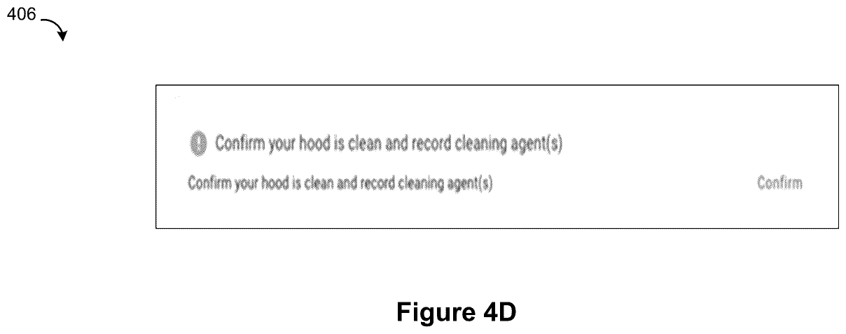

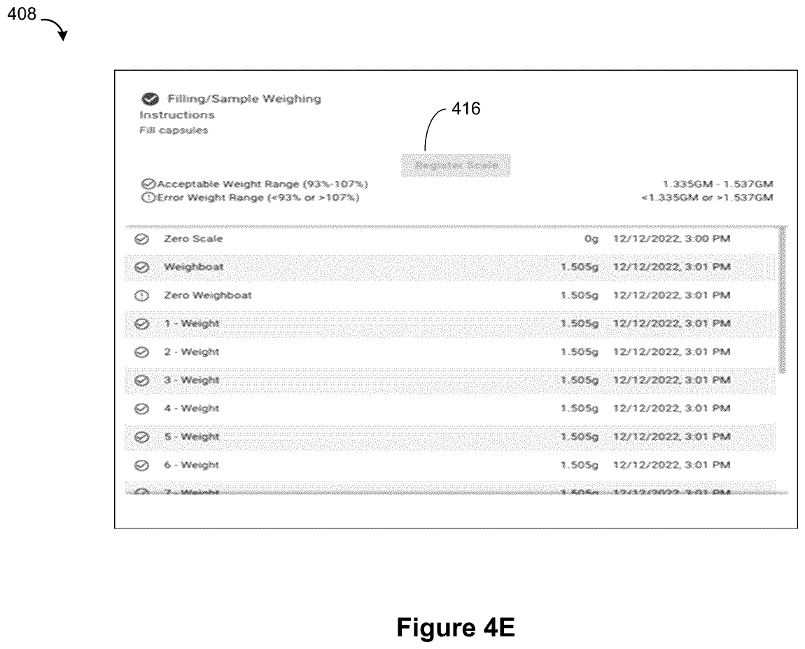

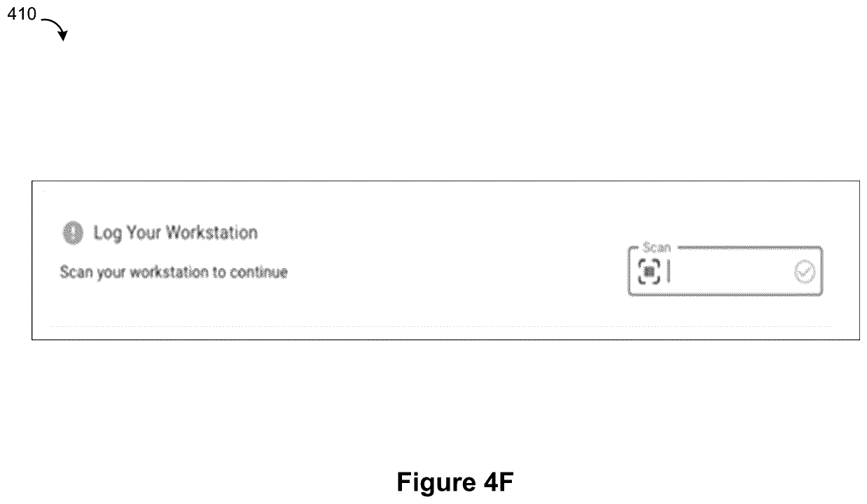

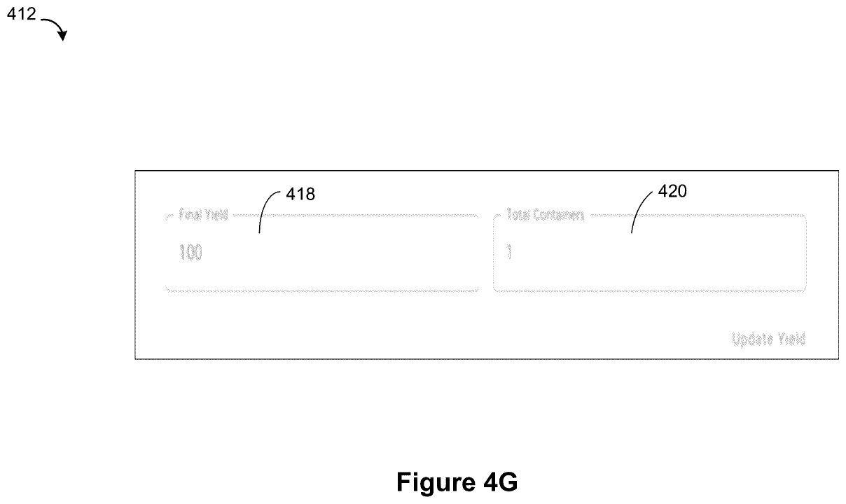

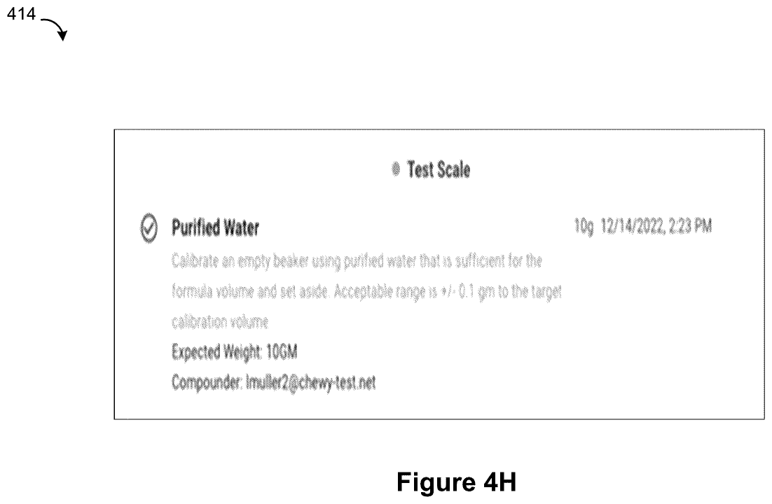

A compounding workflow may generate a label as the workflow progresses. FIG. 4A shows an example label 400, according to some embodiments. The label may be accessed at any time via a task header. As certain steps are completed, more data may be added to the label, such as a beyond use date. The label may include a barcode A that is a task identifier when the lot is a sub-formula, otherwise may be a part number. The label may also include a barcode B which may be a GS1 data matrix containing a task identifier, a part number, quantity, a beyond use date, and/or a start date. This barcode may be consumed by a warehouse management system (WMS). A GS1 data matrix is a two-dimensional matrix barcode which may be printed as a square or rectangular symbol made up of individual dots or squares. The label may also include a barcode C that is a data matrix containing the task identifier to be used in task verification scanning.

A workstation can be rescanned at any time via a task header. The recorded workstation may be added to an equipment audit table. When returning to an in-progress task, a technician may be required to scan a task label to continue. This may help prevent mix-ups and ensure that the technician knows the task to which they are returning. Barcode C described above may be scanned. A compounding addendum is a pharmacist-only record that can be added to after many tasks are complete. Successful equipment scans may be recorded on an equipment audit table that displays entries at the bottom of the compounding workflow. Equipment may include mixers, workstations, and scales.

A list step may include a generic step that can be used to hold any number of sub-steps to be completed in order. An example of a generic step is cleaning. No equipment may be configured. A list step may be a process step in a recipe. For example, a list step may include a step for lifting an object from point A to point B (in a lab). Sub-steps may include confirm instruction, weigh samples, scan workstation, scan mixer, calibrate beaker, container weighing, and ingredient transfer. A step may be considered complete when its sub-steps are completed, and necessary approvals are complete. A weight step is a complex step that allows users to weigh ingredients in any order. This step may require a user to scan a scale before progressing to the ingredient weighing. A sub-step for this step type may be a record weight sub-step for which there will be one per each ingredient in the formula. A final approval step may be functionally the same as the list step. This step may be at the end of each lot task and may mark the lot task as complete when this step is finalized.

A record weight sub-step may be a functionality to record weight for each ingredient. Measurements may be recorded via a scale, while volume and “countable units” may be entered via a text box. A user may start by scanning an ingredient barcode to verify they are on the correct ingredient, and then the step may be marked complete when the recorded weight is within the acceptable ranges. FIG. 4B shows an example user interface 402 for recording a current weight for an ingredient, according to some embodiments. The user may scan an ingredient (e.g., by selecting an affordance 406). The system may respond with a summary and/or details 416 of the ingredient. Subsequently, the user may record weight (e.g., by selecting an affordance 402) for the ingredient. The system may connect to a scale in response, respond with details 408 (e.g., delay for connecting to the scale, expected weight). A sliding scale may be shown with an acceptable range (a lower bound 410, an upper bound 412) and a current weight 414. The user may select the affordance 402 to record the current weight, and/or add the ingredient to a new lot (by selecting an affordance 404). FIG. 4C shows an example user interface 404 for rescanning an ingredient, according to some embodiments. A confirm instruction sub-step may include text and/or a confirmation button. FIG. 4D shows an example user interface 406 for confirming a state of the lab or the interface, according to some embodiments. In this example, the interface asks the user to confirm if the hood is clean and record cleaning agent(s).

A weigh samples sub-step may have users measure samples of a completed batch. The acceptable ranges may be configurable via a formula template. A minimum sample count and/or expected weight may be variable based on dosage form. Dosage forms may include default minimum samples (e.g., 10% total task quantity), default target weight calculation (e.g., (total weight/qty) times tolerances), transdermal minimum samples (e.g., the value 1), transdermal target weight calculation (e.g., total weight times tolerances). Sub-step may be considered valid when the number of recorded values within an expected weight range is greater than or equal to the minimum sample count. The initial 3 measurements may not count towards this goal as they are meant for zeroing the scale. FIG. 4E shows an example user interface 408 for filing and/or sample weighing, according to some embodiments. The user interface may include an affordance 416 for registering a scale, and/or may show details of weighing, including acceptable weight range, error range, zero scale, and/or weighing.

A scan workstation sub-step may be a sub-step that requires a valid workstation to be scanned. FIG. 4F shows an example user interface 410 for logging a workstation, according to some embodiments. A scan mixer sub-step may be the same as the workstation scan except the sub-step may validate that the scanned equipment is a valid mixer. A record yield sub-step may be a sub-step that allows both the pharmacist and technician to mark the final yield and containers for the lot task. FIG. 4G shows an example user interface 412 for recording a yield, according to some embodiments. The example shows fields for final yield 418 and total containers 420, for recording/updating yield. A calibrate beaker sub-step may be a sub-step used by the pharmacy to calibrate the beaker to be used by their task. A technician may fill a beaker with water (1 gram is equal to 1 milliliter) to the target amount and draw a line on it to help with a following weight step. The target weight may be +/−1 gram from the lot task quantity. A technician may register the scale and then record measurements. When a measurement meets the expected weight, the sub-step may be marked complete. FIG. 4H shows an example user interface 414 for calibrating an empty beaker, according to some embodiments. An empty beaker may be calibrated using purified water. An acceptable range, expected weight, and/or compounder name may be displayed.

A container weighing sub-step may behave the same as a calibrate beaker sub-step. This step may require the user to zero the scale first. Rather than continuing until the expected weight is met, this sub-step may accept a second valid weight and store it for later use as an empty container weight. This step may be used in tandem with the next sub-step. An ingredient transfer sub-step may be similar to the container weighing sub-step. This sub-step may be performed after a container weighing sub-step anywhere in the formula. The sub-step may display the transferred ingredient amount as follows: transferred ingredient weight is equal to total weight minus empty container weight, where total weight is the value measured in this sub-step and empty container weight is measured in the previous container weighing sub-step. This value is then displayed to give additional information to the pharmacist who can decide if the lot should be scrapped or not based on the results.

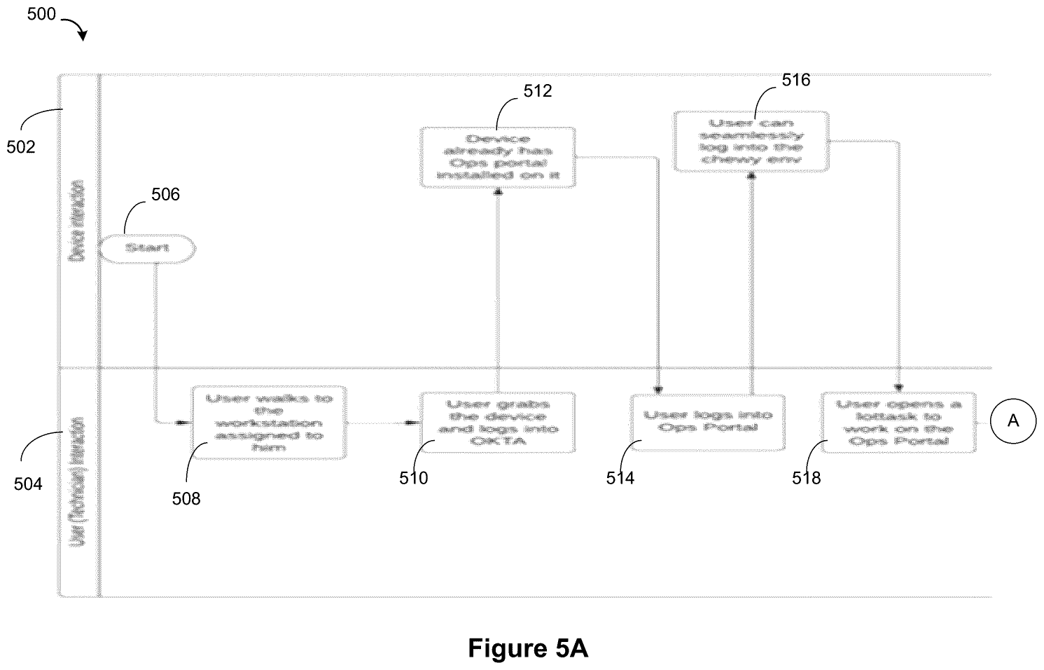

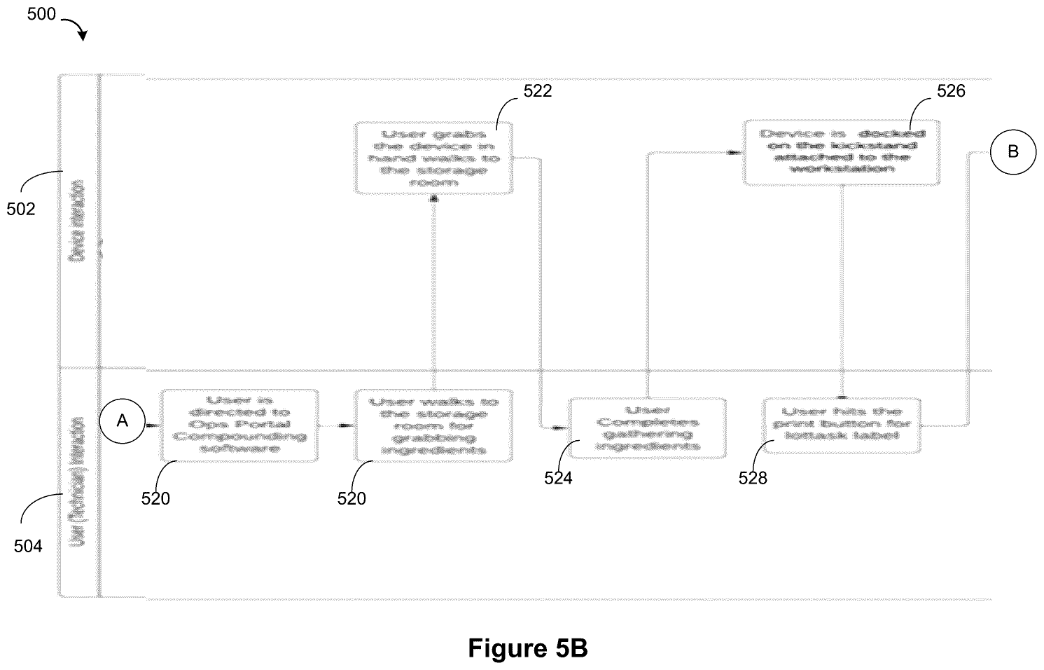

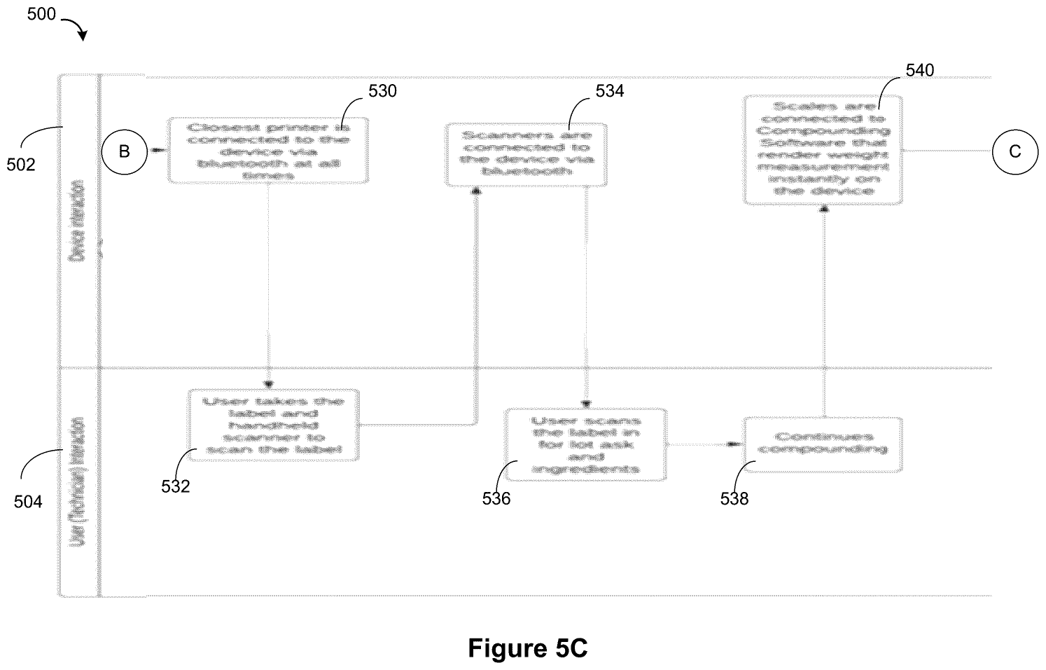

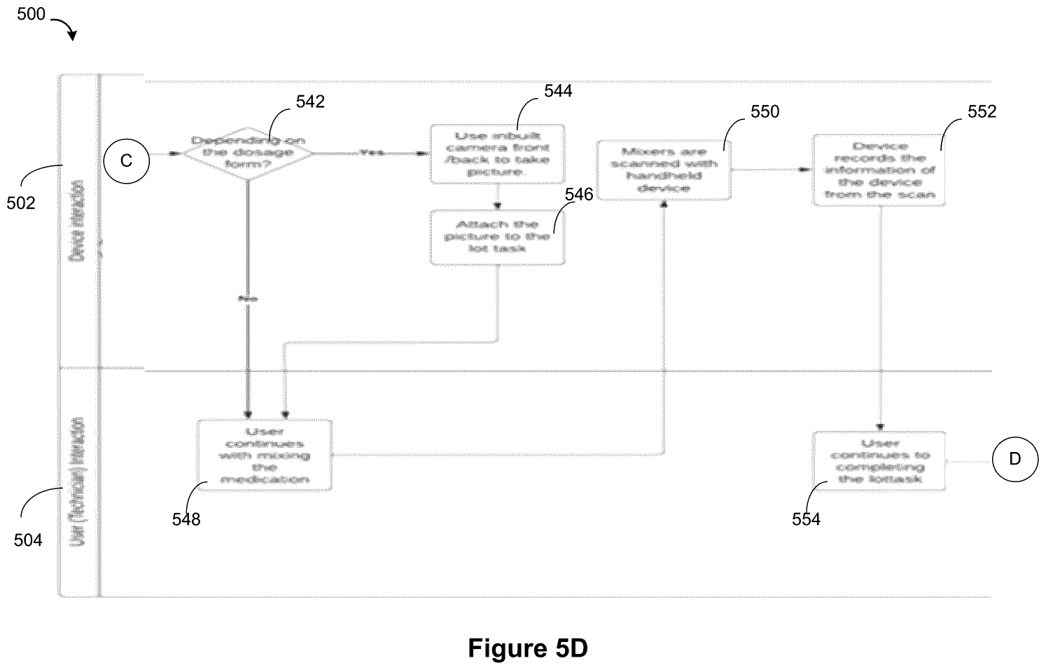

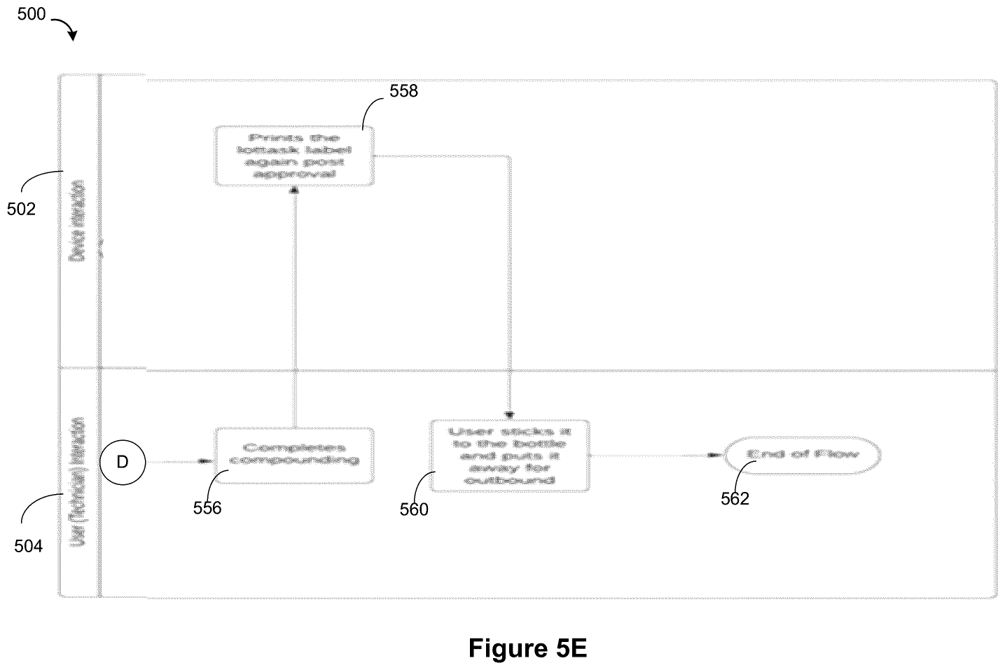

FIGS. 5A-5E show a flow diagram of an example compounding workflow 500, according to some embodiments. Referring to FIG. 5A, the example shows tasks that may be performed by a user (e.g., a technician) interaction 504, and a device interaction 502. The example is shown for illustrating an example workflow, and different task demarcations may be allowed in different embodiments. The workflow may be started (506) via a device interaction. A user may walk (508) to a workstation assigned to them. The user may grab (or reserve) the device and log in (510). The device may already have an installed (512) ops portal (described above), or the portal may be auto installed. The user may log into the ops portal (514). The user may log into a business environment (516). The user open a lot task to work on the ops portal (518).

Referring next to FIG. 5B, the user may be directed to an ops portal compounding software (520). The user may walk to a storage room or area for obtaining ingredients (522). The user may also reserve the device while walking to a storage area (522). The user may complete gathering ingredients (524). The device may be docked (526) on the kickstand attached to the workstation. The user may select a print button for obtaining a lot task label (528). Referring next to FIG. 5C, a closest printer may be connected to the device via a shortrange wireless connection (530). The user may take the label and/or a handheld scanner to scan the label (532). The scanners may be connected to the device via shortrange wireless. The user may scan the label in for a lot task and/or ingredients (536) and/or may continue compounding (538). The scales may be connected to a compounding software that may render weight measurement on the device (540).

Referring next to FIG. 5D, if the lot task depends on a dosage form (condition tested in step 542), the user may use a camera (e.g., an inbuilt camera) (544), and/or attach the picture to the lot task. If the lot task does not depend on the dosage form, the user may continue with mixing the medication (548). In any case, the mixers may be scanned (e.g., using a handheld device) (550). The device may record information from the scan (552). The user may continue to complete the lot task (554).

Referring next to FIG. 5E, the user may complete the compounding (556), print the lot task label post approval (558), and/or prepare the prepared compound for shipping (e.g., sticks the label to a bottle containing the preparation, marks for outbound shipping) (560), which may mark the end of the flow (562).

### Example Demand Processing

Demand processing may create lot tasks based on forecasts, orders, inventory, and/or days of coverage targets. Data for calculations may be populated via several processes including inventory synchronization, forecast import, and order events. Frequency of the demand processing may be predetermined (e.g., every five minutes). Demand processing may be scheduled via a Kubernetes Cron job.

Demand processing may include the following steps. First, formula demand summaries may be fetched. A formula demand summary may contain a view in the data service that summarizes quantities for orders, forecasts, inventory, and lot tasks. The view may also pull in shelf life and days of cover (DoC) targets, which are used in downstream logic. Formula demand summaries may be at the part number level, where a view is created so that a given part should only appear once. Second, available supply may be calculated. Available supply may include unallocated inventory and lot tasks in progress. Unallocated inventory may be calculated as inventory available minus inventory allocated, where inventory allocated contains order items with a status inventory found. The status inventory found means that order item has been released for shipment, but not yet been shipped. Inventory service may not yet reflect decrements for these orders, and thus they must be accounted for to avoid releasing more orders than can be fulfilled by inventory. In addition, lot tasks may be associated with non-terminal status, such as new, pending and review. Based on this information, the demand processing may calculate demand.

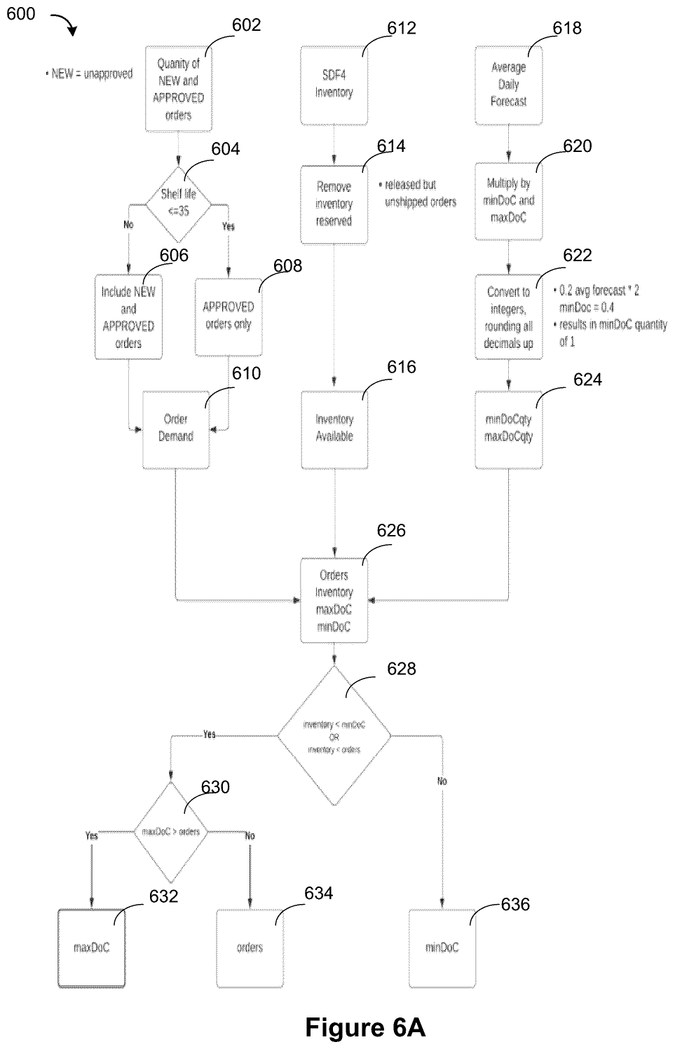

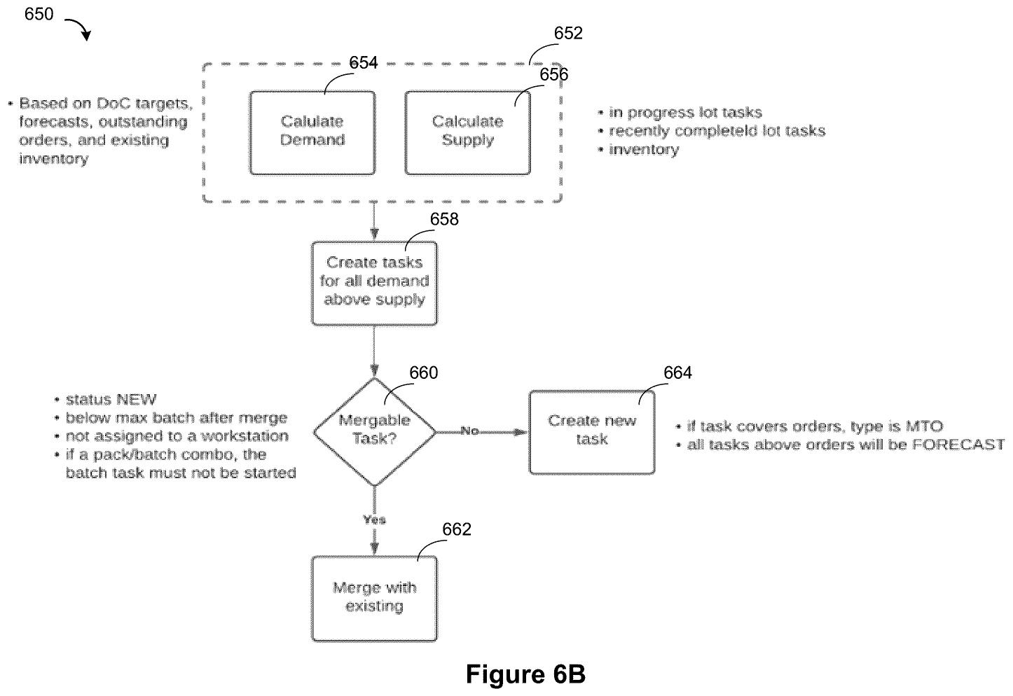

FIG. 6A shows a flowchart for an example process 600 for calculating demand, according to some embodiments. Demand may be calculated using one or more inputs 626, including order demand 610, inventory available 616, and/or days of cover (DoC) quantity 624. DoC quantity 624 may include maximum DoC quantity (referred to as maxDoCqty), and minimum DoC quantity (referred to as minDoCqty). To obtain order demand 610, the system may be configured to check the shelf life. If the shelf life is more than a pre-determined number of days (e.g., 35 days), order demand 610 may include (606) both new and approved orders. If the shelf life is not more than the predetermined number of days, order demand 610 may include (608) only approved orders. Inventory available 616 may be obtained by removing (614) inventory reserved from an inventory 612. Inventory reserved refers to released but unshipped orders. DoC quantity 624 may be obtained by multiplying (620) average daily forecast 618 by maximum DoC 632 or minimum DoC 636, and the resulting numbers of DoC quantity 624 may be further converted (622) to integers, (e.g., by rounding decimals up). For instance, an average forecast of 0.2 multiplying a minimum DoC of 2 would equal 0.4, resulting in minimum DoC quantity of 1 after round-up. The system may be configured to compare (628) inventory available 616 with order demand 610 and DoC quantity 624. If inventory available 616 is smaller than minimum DoC 636 or smaller than order demand 610, the system may be further configured to compare maximum DoC 632 with order demand 610. If maximum DoC 632 is greater than order demand 610, the calculated demand may equal maximum DoC 632. Otherwise, the calculated demand may take the value of order demand 610. If inventory available 616 is neither smaller than minimum DoC 636 nor smaller than order demand 610, the calculated demand is determined by minimum DoC 636.

Days of cover (DOC) targets may be loaded by the pharmacy or business. One goal may be to not make any lot tasks unless inventory falls below the minDoC threshold, or ordered quantity is greater than inventory. In either case, when compounding must be done, tasks may be created to back up to the maxDoC target. If supply is greater than demand, the process ends for the current SKU. If supply is not greater than demand, lot tasks may be created to fetch all order items needing supply and fetch all in progress lot tasks. First, fetching all order items needing supply means to (1) get orders with status prescription approved and new and (2) return any orders with a quantity not met by available supply. For instance, considering a situation where an orderA has a quantity of 2, an order B has a quantity of 2, and lot tasks in progress has a quantity of 2, if order A is covered by the lot tasks in progress, only order B is returned, because older orders are evaluated first. This information is used to categorize new tasks as MTO or FORECAST. After the quantity of new tasks being created covers the order quantity from above, subsequent tasks are FORECAST. This field may be used by, but not limited to, the business. In addition, the information may not give a hint as to the reason the task is originally created and can be used to establish a general pattern. Second, fetching in-progress lot tasks may potentially have their quantity updated if they are mergeable. To become mergeable, a task may be (1) with status NEW, (2) below max batch after merge, (3) not assigned to a workstation, and the batch task must not be started if the task is a combo of pack and batch. Task creation may be limited to 100 tasks per SKU. This is to prevent a surge in tasks if, for instance, a min or max batch size is set incorrectly or there is an unknown bug that leads to demand processing constantly making tasks.

FIG. 6B shows a flowchart for an example process 650 for creating tasks, according to some embodiments. A first step 652 may be to calculate (654) demand and calculate (656) supply, which is based on one or more inputs, including DoC targets, forecasts, outstanding orders, and existing inventory. Those inputs may be obtained from in-progress lot tasks, recently completed lot tasks, or inventory. Then, the calculated demand and the calculated supply may be used to create (658) tasks for all demand above supply. Next, the system may be configured to determine whether tasks are mergeable. In some embodiments, a mergeable task may be (1) with status NEW, (2) below max batch after merge, (3) not assigned to a workstation, and the batch task must not be started if the task is a combo of pack and batch. If tasks are determined to be mergeable, they may be merged (662) with existing tasks. Otherwise, new tasks may be created. In some embodiment, the type of the new tasks may be MTO if they cover orders. Once the quantity of new tasks being created covers the order quantity from above, subsequent tasks are FORECAST.

### Example Forecast Import

Forecasts may be imported each day from an object storage service file (e.g., an S3 file). This procedure may be dependent on lot task ranking and demand processing. The forecast import may be described as follows. The forecast table has weekly forecasts by part and area (e.g. warehouse). A goal of the import process may be to get formula identifiers and the identifier for forecast from the S3 file. Moreover, a forecast may be ignored if the identifier is not a compound identifier. Furthermore, if there may be an existing forecast for that part and week, the current row may be updated. Otherwise, a new row may be created.

### Example Inventory Synchronization

An inventory synchronization may be a job that runs at predetermined intervals (e.g., every 5 minutes) to synchronize the item inventory table with inventory quantities from an inventory service. Frequency of the inventory synchronization may be every five minutes and may be scheduled via a Kubernetes Cron job.

The inventory synchronization may include the following steps. First, a database may store an inventory for a given part in an item inventory table. This information may be used to determine whether lot tasks need to be created (e.g., referred to lot task demand processing) and order can be released (e.g., referred to order releasing). Moreover, a part number's inventory may be fetched if there are any outstanding orders or forecasts. Furthermore, part numbers may be fetched through an inventory sync endpoint (e.g., referred to as /v1/inventory/availability/{partNumber}). Multiple warehouses may be supported. In addition, inventory sync inventory may have a built in 100,000 base amount for compound items such that the items do not show as out of stock. For instance, if inventory synchronization reports 100,000 and there may be actually 0 in the inventory table, then there may be 5 in the inventory table when inventory synchronization reports 100,005.

### Example Formula to Lot Task Cascading

A procedure for formula to lot task cascading may be used to make changes to the formula which may include the template, steps, process details and then cascade the changes to the yet to be started on lot tasks. A component or formula may indicate a normal lot task that uses single ingredient, while a sub-formula may indicate a normal lot task that uses multiple ingredients. A pack task may indicate the list of lot tasks under the expandable sub-formula. Formula changes to lot task cascading may occur on a user interface view of the lot task.

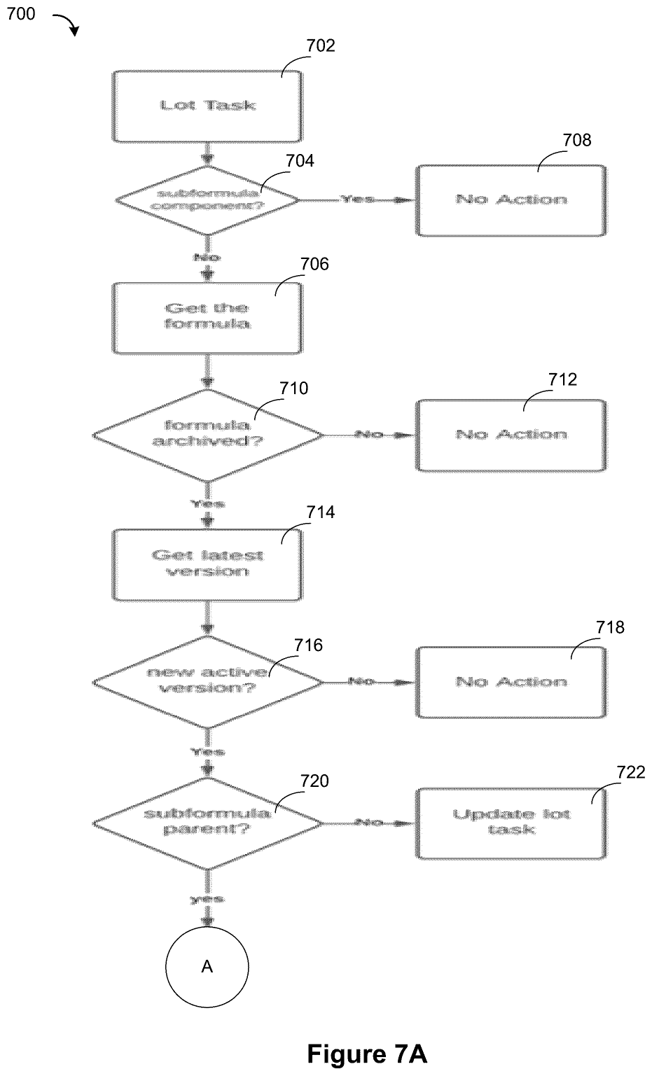

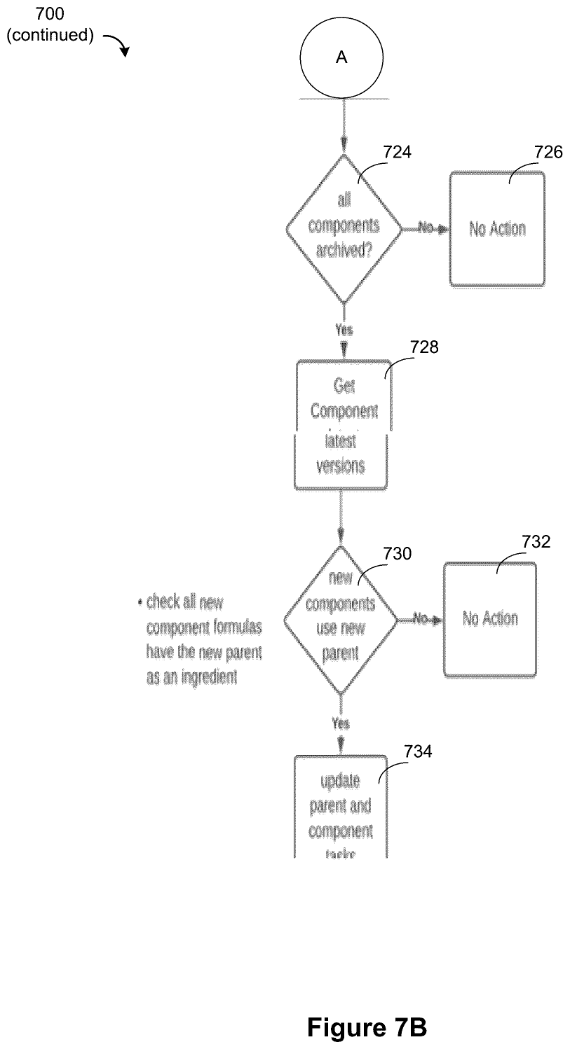

FIGS. 7A and 7B show a flowchart of an example process 700 for updating formula to lot task cascading, according to some embodiments. After a lot task 702 is assigned, the system may be configured to check whether the lot task is attached (704) to a sub-formula parent. If the lot task is attached to the sub-formula parent, no action 708 may be required. If the lot task is not attached to the sub-formula parent, the formula may be obtained (706) and then attached to the task. Then, the system may be configured to check whether the formula is archived (710) and there is a new active version. If the formula is not archived, no action 712 may be required. Otherwise, the formula is standalone (i.e., the lot task is not attached to the sub-formula parent and there is no sub-formula ingredient), and the latest active version would be obtained (714) and then attached to the lot task. Next, the system may be configured to check whether the sub-formula parent has a new active version 716 with new sub-formula ingredients. If not, no action 718 may be required. Otherwise, the system may be configured to check whether all components have a new active version with the new sub-formula parent. If not, sub-formula parent and their components may be updated to point to the new versions. Otherwise, an ingredient may be found and recognized.

Referring next to FIG. 7B, the system may be configured to check whether all components that use single ingredients are archived (724). If not, no action 726 may be required. Otherwise, the latest versions of components may be obtained. Then, the system may be configured to check (730) whether new components use new parents. In some embodiments, the system may be configured to check whether all new component formulas have new parent as an ingredient. If it is determined that new components do not use new parent, no action 732 may be required. Otherwise, the parent and component tasks may be updated accordingly.

In some embodiments, the following logic may be performed to get the result. For instance, the system may be configured to look for latest (by active), as previous versions if any, are archived upon a new version creation. An example of the logic is shown below:

```
public class FormulaCascadeResult {
  Formula updatedFormula;
  LotTask;
  List<LotTask> updateEligiblePackTasks;
  Map<UUID, Formula> updatedFormulasByIdentifier;
}
```

In some embodiments, if applicable, the lot task may be updated with a new formula and a formula identifier (e.g., referred to as updateFormulaForLotTask). Accordingly, relevant child lot tasks, if any, may be updated simultaneously. It may be unlikely that if updates were to occur, they would cause issues, because it is a rare scenario that there would be new versions for archived formulas. On the other hand, lot task assignment may have issues with new versions if the dosage form or active pharmaceutical ingredient (API) is changed. In addition, if a sub-formula parent update cascades correctly, it would be rare to have the parent formula updated. This may not apply to a component update. Such cascading procedure for updates may limit the state where there is an after workstation assignment of active parent but archived component. In some embodiments, an exception may apply to the case where the component is archived from the user interface with no new version created, which would be always a possibility for any lot task.

### Example Lot Task Ranking

A procedure of lot task ranking may give all lot tasks a ranking, which is used to prioritize work in the lab. Ranking factors may be configurable on a user interface and stored in a ranking factor table. Ranking factors may be also configurable in the code (i.e., any updates require a deployment). The frequency of updates on the lot task ranking may be on demand via a user interface. For instance, updates may occur at the top of the hour via a Kubernetes Cron job. In some embodiments, each lost task may be updated individually or in bulk. During the procedure, only pack/component tasks are directly ranked, while sub-formula tasks may be ranked based on the highest ranked component.

Each batch of tasks may be allotted a specific ranking determined by a metric that may be used to establish priority levels of tasks conducted within a laboratory setting. The parameters governing the assignment of these rankings may be fully configurable within the codebase. It is important to note that any adjustments or enhancements in this regard may necessitate a subsequent code deployment to take effect. Task initiation may be a twofold process: it is either prompted by a user request through the interface, affording on-demand control, or automatically triggered at the commencement of every hour through a meticulously orchestrated Kubernetes Cron job.

A ranking schema may be applied to pack/component tasks, where the ranking impacts task sequencing. Intriguingly, sub-formula tasks may follow a distinct protocol; their ranking may be intricately tied to the highest rank accorded to any constituent component within the overarching formulation. This complex interplay may ensure that the hierarchy of sub formula tasks resonates with the hierarchy of their encompassing components.

The lot task ranking may be determined by multiple ranking factors and each ranking factor may be associated with a weight. The ranking factors may be divided into multiple categories, including normalized factors (or referred as percentage weightings), flat ranking modifiers, and sliding flat modifiers. In some embodiments, the category of normalized factors may include multiple factors, including trailing 30 day number of orders, pending order count, days of cover (DoC) delta, and maximum time since order placed (MTSO). Trailing 30 day number of orders may indicate number of orders being shipped within 30 days. This factor may be used to prioritize fastest movers. Trailing 30 instead of trailing 60 may be used to minimize the effect on new SKUs. Pending order count (also referred to as total number of orders) may indicate number of pending orders (i.e., orders in a non-terminal state). Days of Cover (DoC) delta may indicate the difference between DoC on hand and a target DoC amount, which is set at the SKU level. This factor may be normalized by comparing the target and on hand DoCs instead of some math equations. Maximum time since order placed may indicate passage of time since a customer placed an order. The maximum time may be set to 200 hours to exclude outliers. This factor may be used to prioritize the oldest orders to minimize click to ship time. In addition, this factor may be in place so that SKUs with unapproved orders are weighted higher than other orders and are worked prior to approval. In some embodiments, the category of flat ranking modifiers may include covers approved order, MTO refrigerated, pending tasks, and approved order age tier. Covers approved order (i.e., MTO) may indicate a task that covers an approved order. MTO Refrigerated may indicate a task that covers an approved order and is also refrigerate. Pending tasks (i.e., pre-weighed) may indicate a task that has a weight step completed. Approved order age tier may stratify MTO orders based on age, so that older approved orders needing awaiting tasks completion are worked first. In some embodiments, Approved order age tier may be further divided into four tiers based on sliding flat modifiers: (1) over 150 hours; (2) over 100 Hours; (3) over 75 Hours; and (4) less than or equal to 75 hours. To be precise, the tier with over 100 hours may include orders between 101-150 hours old. The same methodology may apply to all tiers.

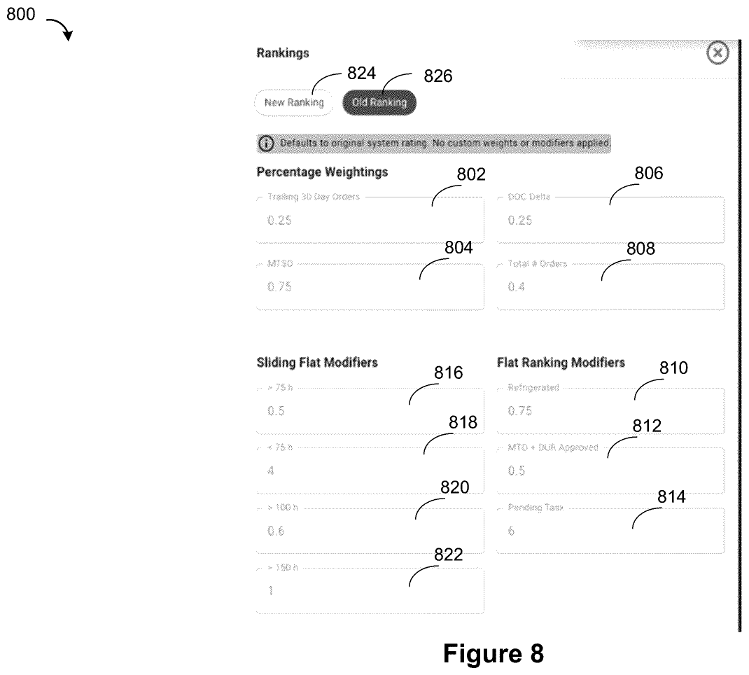

The values of each ranking factor may be configured via a user interface and the data may be stored in a table for ranking factor. FIG. 8 shows an example user interface 800 for configuring ranking factors, according to some embodiments. The user interface may provide affordances for configuring percentage weightings, e.g., trailing 30 day orders 802, MTSO 804, DOC delta 806, and total number of orders 808. The user interface may also provide affordances for sliding flat modifiers, e.g., greater than 75 hours 816, less than 75 hours 818, greater than 100 hours 820, and greater than 150 hours 822. The time durations may be obtained from operations. The user interface may also provide affordances for flat ranking modifiers, including refrigerated 810, made-to-order (MTO) and drug utilization review (DUR) approved 812, and pending task 1314. Some embodiments provide multiple ranking options, e.g., a new ranking and an old ranking. FIG. 8 corresponds to an old ranking.

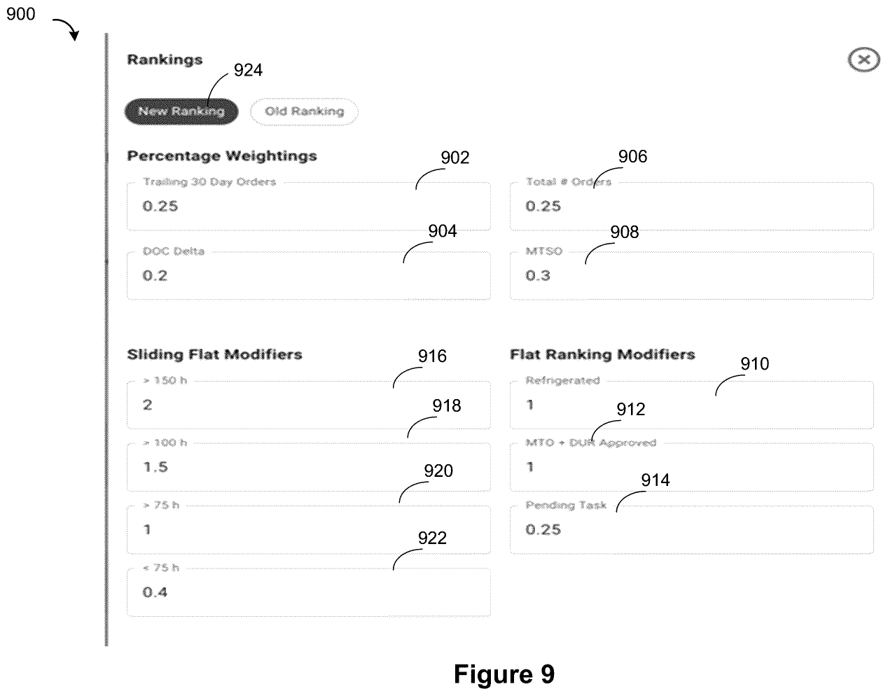

FIG. 9 shows another example user interface 900 for configuring ranking factors, according to some embodiments. This interface is similar to the example in FIG. 8. This example may correspond to a new ranking 924. The user interface may provide affordances for configuring percentage weightings, e.g., trailing 30 day orders 902, MTSO 908, DOC delta 904, and total number of orders 906. The user interface may also provide affordances for sliding flat modifiers, e.g., greater than 150 hours 916, greater than 100 hours 918, greater than 75 hours 920, and lesser than 75 hours 922. The user interface may also provide affordances for flat ranking modifiers, including refrigerated 910, MTO and DUR approved 912, and pending task 914. The order of these affordances (and the corresponding weightings and/or modifiers) may be changed (automatically and/or by a user), as illustrated in FIG. 9 (relative to FIG. 8).

In some embodiments, processing ranking factors may require normalization. Ranking factors that are not naturally between 0 and 1 may be normalized across parts. A purpose may be to ensure that factors use a common scale across all part numbers so that further comparisons are valid. Normalized value of a ranking factor may be calculated as: normalized value=(value−minimum)/(maximum−minimum). For instance, if the actual value of a ranking factor is 10 and the allowed minimum and maximum values of the ranking factor are 3 and 17, respectively, the value of 10 becomes (10−3)/(17−3)=0.5, the value of 3 becomes (3−3)/(17−3)=0, and the value of 17 becomes (17−3)/(17−3)=1.

Lot task ranking may include the following steps. In a first step of the lot task ranking, the system may be configured to refresh all the inputs that will dictate the ranking. This data may be considered as a ‘factor input.’ In some embodiments, calculating approved MTO refrigerated require mapping the formula identifier to a boolean, which indicates whether the SKU has an approved order and is refrigerated.

In some embodiments, calculating approved order quantity may require mapping the formula identifier to the approved order quantity, which accounts for existing inventory and recently completed tasks to avoid unnecessary ranking bumps. The formula may be described as quantity to cover approvals=order quantity prescription approved-available inventory+order quantity found−recently completed tasks, where order quantity inventory found is reserved inventory and recently completed tasks are tasks completed, but potentially not yet in inventory.

In some embodiments, calculating shipped order quantity may include mapping the formula identifier to the shipped order quantity, normalized across all parts.

In some embodiments, calculating pre-weigh completed may require mapping the lot task id to check whether or not the task has a completed weight step.

In some embodiments, calculating approved order age tier may require mapping the formula identifier to another map of the ordered quantity in each approved order age tier. Example data for the formula identifier for calculating approved order age tier may include over 150 hours, has an ordered quantity of 30, over 75 hours, has an ordered quantity of 40, and/or less than equal 75 hours has an ordered quantity of 100.

In some embodiments, calculating pending order count may require mapping the formula identifier to the number of outstanding orders (not terminal status), normalized across all parts.

In some embodiments, calculating days of cover delta may not need normalization because it is naturally between 0 and 1. For instance, if target quantity is 100 and there are 75 in inventory, the gap would be 0.25. In another instance, if there are 101 in inventory, the gap would be 0. If target days of cover is 0, a value of 0 would be returned for this factor, which indicates that the pharmacist is expecting the part to be MTO only. To calculate days of cover delta, a first step may be to get the next 5 weeks forecasts and covert it to units consumed per day. The system may be configured to (1) for each weekly forecast, get the units per day by dividing by 7, (2) return the average of the number above across weekly forecasts, and (3) calculate total required by multiplying units per day times the DoC target. Short shelf life SKUs may be defined as not greater than 30 days by using a DoC target of 3. With respect to all other SKUs, the target may be 30 days. A second step may be to calculate the DoC adjustment per unit. This value means how much of the DoC gap is filled when one unit is completed. For a given part, associated tasks may have a decreasing DoC Delta. For instance, suppose there are tasks Task A and Task B. Task A will have a higher DoC Delta factor if it is ranked before Task B. The goal is to rank Task B as if Task A were completed, and thus some of the DoC gap will have been filled. Equations are given as follows: (1) units per day=average (forecast 1/7, forecast 2/7, etc.), (2) total required=units per day times 30, (3) document gap=1−(current SDF4 inventory/total required), (4) adjacent per unit=1/total required, and (5) each lot task quantity drops the docs gap by adjacent per unit times task quantity. For instance, if total required=100 and inventory=75, doc gap=1−(75/100)=0.25 and adjacent per unit=1/100=0.01. Accordingly, if the quality of tasks is 25, each lot task quantity drops the docs gap by 0.01*25=0.25, meaning the DoC gap is gone after 25 units.

In some embodiments, weights by factor are the factor values stored in the database.

In some embodiments, calculating maximum time since order may require mapping the formula identifier to the maximum time since an outstanding order was placed, normalized across all parts. If the SKU has inventory coverage, this factor would be 0.

In some embodiments, demand data may wrap supply and demand for SKU. Demand data may have data, such as inventory, lot tasks in progress, ordered quantity, and forecasted quantity.

In the second step of the lot task ranking, the system may be configured to get lot tasks with status new, pending, and review. Sorting may not be specified, and hence the data-service would default to ascending order by creation time. In a third step of the lot task ranking, the system may be configured to rank each task.

In some embodiments, to obtain covers approved order, the system may be configured to, for the current part, compare total amount of lot tasks already ranked against the approved order quantity from the first step. If total ranked is still below the total approved, the system may be configured to add covers approved order value stored in the database. The equation may be calculated as covers order approved quantity minus total ranked quantity greater than zero.

In some embodiments, to obtain refrigerated, the system may be configured to add the MTO refrigerated value stored in the database if the task is MTO (i.e., covers an approved order from above) and the data from the first step of the lot task ranking indicates a refrigerated part.

In some embodiments, to obtain shipped order count (i.e., trailing 30 day order count), the system may be configured to multiply the normalized trailing 30 day order count from the first step of the lot task ranking times the value stored in the database.

In some embodiments, to obtain pre-weighed, the system may be configured to add the pre-weighed value stored in the database, if the data from the first step of the lot task ranking indicates this task was pre-weighed.

In some embodiments, to obtain pending order count, the system may be configured to multiply the normalized pending order count from the first step of the lot task ranking times the value stored in the database.

In some embodiments, to obtain approved order age tier, the system may be configured to input approved order quantities that are stored by approved order age tier during the first step of the lot task ranking. Tiers may be prioritized based on the factor values. For instance, over 150 hours has the highest value, and thus orders in this tier are examined first when assigning a rank to a task. As an example, suppose Task A quantity is 40, Task B quantity is 40, Task C quantity is 10, and over 150 hours is 25 and over 100 hours is 50, Task A may cover a quantity of 25 ordered in over 150 hours, and hence the ranking is bumped by the value stored in the database. Task A may cover a quantity of 40, and thus it already covered some of the next tier and accordingly over 100 hours is reduced to 35. Task B may cover the 35 remaining in over 100 hours, and thus the ranking is bumped by the value stored in the database. All tiers may be accounted for, and Task C gets no modifier for this factor.

In some embodiments, to obtain days of cover delta, the system may be configured to get the days of cover factor from the first step of the lot task ranking and subtract from this factor the quantity of any tasks already ranked for the current part times the doc adjustment per unit. Accordingly, adjusted DoC delta may be calculated as adjusted DoC delta=doc delta normalized-(in progress ranked quantity times adjust per unit). Then, adjusted DoC delta may be multiplied by DoC delta stored in the database.

In some embodiments, to obtain maximum time since order, the system may be configured to multiply the normalized maximum time since order from the first step of the lot task ranking times the value stored in the database.

In a last step of the lot task ranking, the system may be configured to update sub-formula task ranks by finding the maximum rank for component, child, or batch tasks of the sub-formula and setting the sub-formula task rank to be minimally higher than this maximum.

### Example Formula Management

Formula management is a component of compounding creation workflow. Formula management may encompass step by step instructions for compounders to convert raw materials into finished products that are dispensed based on customer orders.

| Term | Definition |
|---|---|
| Template | Each formula has a set of steps and sub-steps which may be common across |
| | multiple formulas. Template is a quick starting point that allows users with the |
| | required access to configure those ready to use template without starting from |
| | the scratch each time they create a new formula. |
| Formula | To compound finish product to be dispense to our customer, we need formula |
| | for each of the SKU. Formula is the step by step instructions to make the |
| | finished product from raw ingredients. Examples of Formula |
| | 1. Tylosin Tartrate Capsule 75 mg. |
| | 2. Pimobendan Oral Liquid 30 mg/mL Chicken-Marshmallow- 90 mL |
| | Note: The above examples include Active ingredient API (Pimobendan), Dosage |
| | form (Oral Liquid), Strength (30 mg/ml), flavor (chicken-marshmallow) and |
| | pack size (90 ml) |
| | In case of Parent Formulas, they are made in bulk that do not map to a customer |
| | facing SKU such as |
| | Sildenafil Oral Liquid 2-mg/mL Chicken (notice there is no pack size) |
| Lot task | Lot Tasks are unique batches of a Compound produced for a specific formula. |
| Steps | Each self-contained step of execution - Example: Cleaning the workstation, |
| | Weighing Ingredients, Approvers check, Mixing, Moulding, Filling. |
| | Steps have a set of global parameters - such as being enabled for Notes, Begin |
| | button, Capture of photo, Compounder, Approver |
| Sub- | Sub-Steps are actions that are to be performed with in the step, such as read |
| Steps | instructions, weigh ingredients, weigh sample, confirm the completion of an |
| | action, etc. |
| QS | Quantity Sufficient |

Formula management is one of the module within compounding software to ensure that processes are followed in during compounding creation workflow to safeguard quality and consistency across all finished products compounded with in the lab.

At a high-level, formula management module may include the following workflows: template configurations, formula configurations, bulk uploads, and version control and audit logs.

### Template Configurations Functionality

Formula management workflow may begin with pre-requisites to house the configurations as a template which may be leveraged downstream to expedite formula configuration workflow. Template configurations may allow the users with the valid permissions to add a new template or clone an existing template to customize them with the steps that will be used to build a formula.

To begin with, users may assign a name to a template along with description and steps. These steps are sequential step by step instructions (such as log in to workstation, cleaning the station, weighing empty jars, measuring ingredients, capturing photos etc.). This may also be used to track when the compounding process begins, if a compounder is required to capture an image for relevant steps, add any notes and a flag to request an approval.

Template may be saved in four status throughout its lifecycle which are categorized as ‘Draft’ (to be continued to work upon later), ‘Review’ (pending approval), ‘Active’ (to leverage in building formulas) or ‘Archived’ (no longer used in the lob). When pharmacist is done with editing the formula template, they may flag them as “Pending Review” status to be reviewed by another Pharmacist before it is activated.

Template status and formula status may always be coordinated to ensure as the templates are updated formulas are also updated if application. When a template is updated for an active formula, it creates a new version of the formula, with the updates from the template (new steps/sub-steps, updates to existing steps) and set the new version to active status. When a template is updated for a pending review Formula, it may update the formula with the updated template and maintain the ‘Review’ status. When a formula is in draft, it may keep them in the same status and push the updates from the template.

### Example Formulas Creation and Management

Pharmacists may create new formulas directly in formula management module simply by clicking ‘Create formula.’ Create formula may allow them to add Formula Name, part number, Oracle identifier to create Master formula record. During this process, they may add up to 5 Active Pharmaceutical Ingredient (API) and strength (and Unit of Measurement (UOM) along with expected dosage form, flavor, pack size, beyond use date, route of administration and total quantity expected to be compounded when the tasks are created for these formulas. For items that require special handling such as ‘Refrigeration’ or ‘Hazardous,’ the special handling may be annotated in this setting. Users may also select canned list of instructions to be printed on the ‘Auxiliary label.’ Once created, each formula may be set up to include minimum and maximum days of coverage (DOC), along with min. and maximum batch size. Formula management may provide an ability to manually add ‘Tasks’ by specifying the quantity for a given formula.

### Example Bulk Upload Functionality

Bulk upload may allow pharmacists to leverage Microsoft Excel to upload multiple formulas simply by clicking a button in CS. This operation may significantly improve the time it takes to add formulas into CS while ensuring that the quality is not compromised by maintaining the underlying validation criteria. Similar to manual formula creation, bulk upload may also require a second pharmacist approval before marking those formulas as ‘Active.’ During upload, CS may validate any missing criteria and results in a warning to inform the users whether the bulk upload is ‘Success’, ‘Partial Success’ or ‘Fail.’

Success may mean that all the rows are processed successfully. Partial Success may be classified as some rows that are imported as formula while some failed to create formula. There may be reasons due to which an upload may fail such as having invalid or duplicate identifiers, Formula ID/Part Number, formula name or missing required data. Failed is usually caused when no rows are imported, and no formulas were created.

Show the result file against the row of the input file. The result file may have as many rows as the input file, it may have a few additional columns that contain system generated data such as-created by, date, approved by, version number.

### Example Version Control

After a formula is marked as ‘Active’ for the first time, it may be by default set as version 1. Additionally, CS may allow them to make changes to an active formula and published them once the changes are finalized. Every time a formula is updated, it may be assigned with a newer version while retaining the old version to ensure it can be audited, if needed in the future.

Retired formulas may be accessible in a separate tab within ‘Archived Formula’ for auditing purpose.

### Example Sub-Formula Task Creation

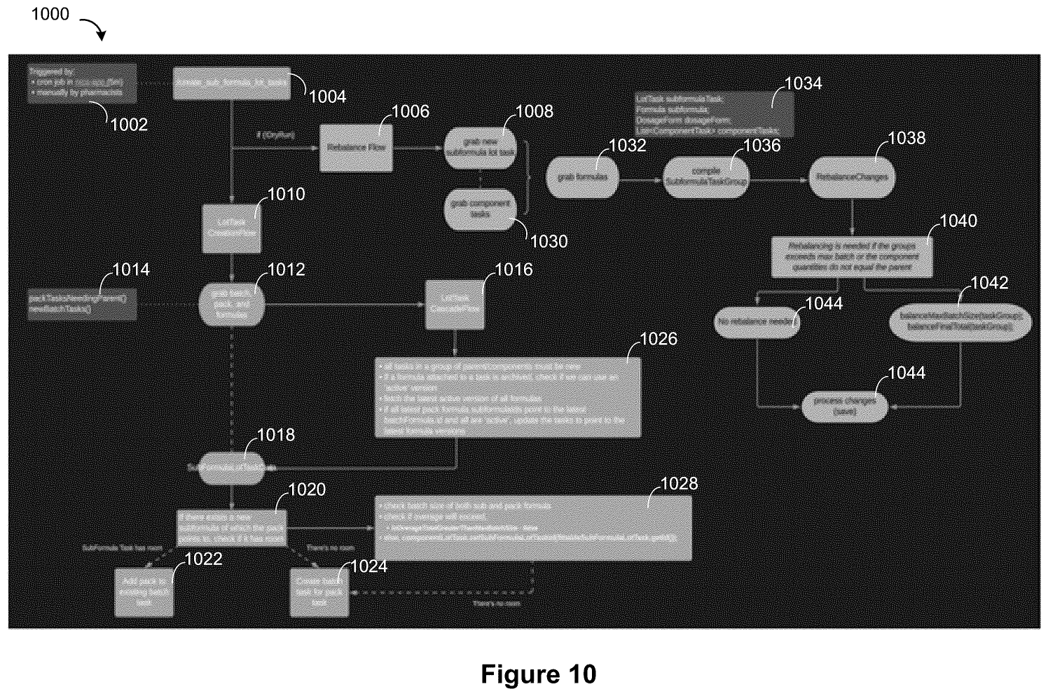

FIG. 10 shows a flow diagram of an example flow 1000 for sub-formula task creation, according to some embodiments. The flow may be triggered by a daemon job and/or may be manually started by pharmacists (1002). A daemon job (e.g., in a prescription application) (1002) may occur every 5 minutes or may be manually triggered by the pharmacists. During a lot task creation flow, there may be a flow known as the cascade flow in which formulas (batch and/or packed) may be cascaded and/or updated, if applicable. From the cascade flow, the data may be compiled and returned back to the lot task creation flow. There may exist sub-formula lot task(s) for lot task already, in which case the system may add to it. If there are no matching sub-formula lot task for a lot task, the system may generate a new task. This may in turn trigger a lot task creation flow step 1010, and/or a rebalance flow 1006 (for rebalancing lot tasks, e.g., across workstations). If rebalance is triggered, a new sub-formula lot task may be processed (1006), which may in turn grab or get hold of component tasks (1030). Under a lot task creation flow (1010), a batch, pack and/or formulas may be obtained (1012). A new batch task may be executed (1014). A sub-formula lot task may be started (1018). A lot task cascade flow may be started (1016). As shown in step 1026, tasks in a group of parent/components may need to be new. In some embodiments, if a formula attached to a task is archived, the system determines if it is possible to use an ‘active’ version. If so, the system may fetch the latest active version of formulas. In some embodiments, if latest pack formula or sub-formula identifiers point to the latest batch formula identifier and are ‘active,’ the system may update the tasks to point to the latest formula versions. A sub-formula task is a parent task that may be applicable for a handful of dosage forms. A pack task is a child task; multiple child tasks may be associated with one parent task. A batch task is a regular lot task that does not conform to this parent-child methodology, e.g., capsules do not have a sub-formula task. For a sub-formula lot task, some embodiments determine (1020) if there exists a new sub-formula of which the pack points to and if so, if the pack has room (for expansion). If the s sub-formula task pack has room, the pack may be added (1022) to the existing batch task. If not, a batch task may be created (1024) for pack task. Some embodiments check batch size of both sub and pack formula, and/or check if overage will exceed. Overage may be preconfigured in a lot task, predefined in a system. For example, compound A includes ingredient X plus 4 ml. Overage limit may be +/−4 ml per bottle, rounded up to nearest 20 ml; SC is +/−2 chews per 45; Td is +/−1 ml per pen. Caps and tabs may not have the sub-formula solution applied.

In some embodiments, rebalancing a flow (1006) may result in grabbing a new sub-formula task (1008) which may in turn lead to a component task being grabbed (1030). These steps may in turn result in grabbing formulas (1032). This may be followed by compiling a sub-formula task group 1 (1036), followed by rebalancing changes (1038). The rebalancing may be needed if the groups exceeds batch, or the component quantities do not equal the parent (1040). Either no rebalance may be needed (1044) or balancing may be effected (1042). Any process changes may be saved (1044).

### Example Sub-formula Task Rebalancing

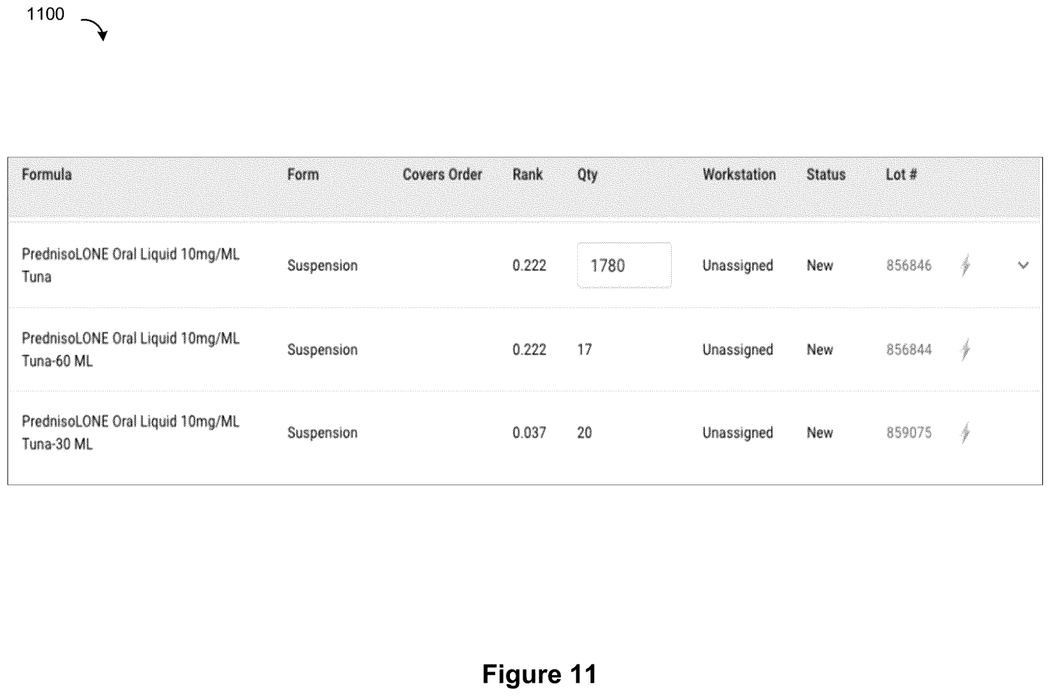

FIG. 11 shows an example user interface 1100 for sub-formula task rebalancing, according to some embodiments. A daemon job may ensure a sub-formula parent batch task is balanced relative to its component or pack tasks. Related processes may include sub-formula task creation pairs pack tasks with batch tasks. Demand processing may update the pack task quantities, causing the need for rebalancing. Frequency for the daemon job may be every 5 minutes via a Kubernetes cron job. Sub-formula may be a formula that is used as in ingredient in another formula. The related tasks may be referred to as a sub-formula, parent, or batch tasks. A pack formula may include formulas that directly map to a part number. Techniques are described herein with respect to formulas that have a single ingredient, and that ingredient points to the sub-formula. A sub-formula task may have one or more associated pack tasks. A sub-formula task may be completed first, and the resulting batch may be used to fill pack tasks. These task groups are visible on a user interface (e.g., the interface 1100 shown in FIG. 11) for any task that is expandable. In the example shown, 1780 ml for task 856846 is used to fill tasks 856844 and 859075. 1780 ml=(17 times 60 ml)+ (20 times 30 ml)+ (4 ml overage per bottle times 37), round up to nearest 20 ml. 1780 ml=1020+600+148, rounded up to nearest 20 ml. A completed flag on the lot task table may be set on the sub-formula task group as a whole. So, in the screenshot shown in FIG. 11, tasks may need to either be approved or cancelled.

The rebalancing process may include obtaining process data, such as assigned sub-formula tasks in status new, associated pack tasks that are not cancelled, associated formulas for all tasks, and/or associated formula ingredients for pack formulas. Some embodiments transform the above data into a list of objects that groups the sub-formula task with its respective batch tasks and includes all formula and formula ingredient information. Sub-formula task groups may be excluded if any of the following conditions is true: if it is not possible to find the associated formula for the sub-formula task, the sub-formula is missing data in a formula process table, the sub-formula minimum/maximum batch is missing or if it is less than or equal to 0, the dosage form does not support automation, cannot find the associated formula for any pack task, any pack task formula has more than one formula ingredient, these formulas are expected to have one ingredient (i.e., the sub-formula), any pack formula is missing data in the formula process table, any pack formula is already above the sub-formula maximum batch which may indicate that this is likely a misconfiguration and they need to be skipped because the rebalance logic would disconnect the pack formula, but it would be picked up again in the main sub-formula task creation. It would then be disconnected again in the rebalance job, creating a continuous loop.

Some embodiments transform the above data into an object that wraps sub-formula tasks that need quantity updates, and pack tasks that need to be disconnected from their current sub-formula batch task. In some embodiments, if the pack task quantities with overage exceed the sub-formula maximum batch, or the sub-formula task quantity is out of alignment with its pack tasks, pack tasks are removed until the sub-formula task is back under or equal to its max batch. The smallest pack task may be removed first, so it has the best chance of being paired with a new existing sub-formula task. Some embodiments calculate the new quantity of the sub-formula task based on the remaining pack tasks. Some embodiments make necessary updates to quantities and pack tasks based on previous step.

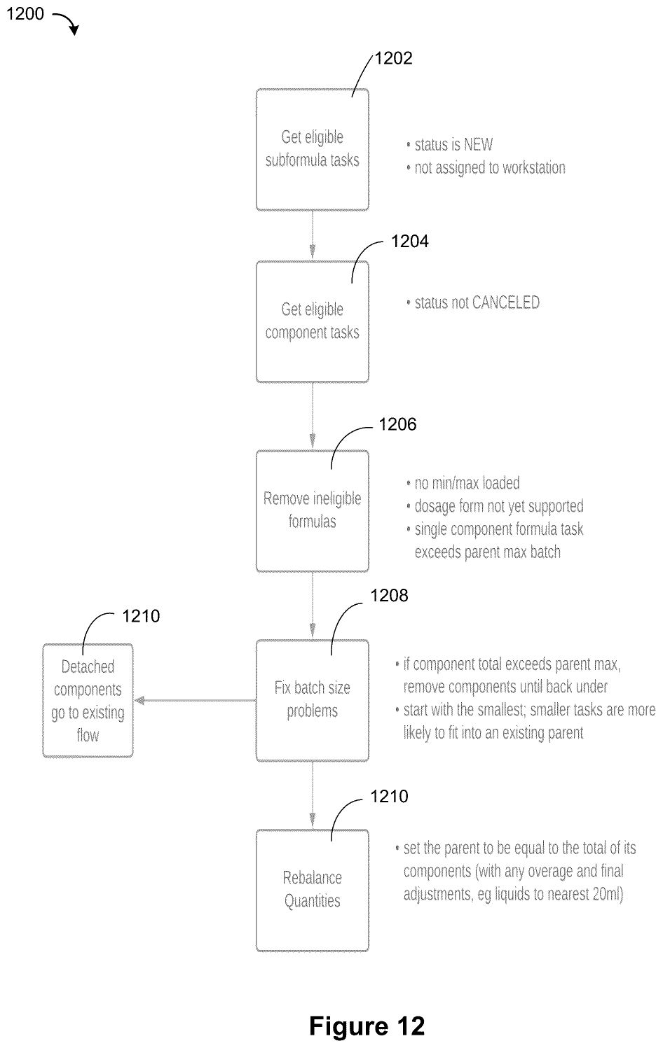

FIG. 12 shows a flow diagram of an example process 1200 for sub-formula task rebalancing, according to some embodiments. Some embodiments obtain eligible sub-formula tasks (1202) (the status may need to be new and not assigned to any workstation), obtain eligible component tasks (1204 (status may not be cancelled), remove ineligible formulas (1206) (e.g., no minimum or maximum loaded, dosage form, single component formula task, formulas that exceed parent maximum batch), fix batch size problems (1208) (e.g., if component total exceed parent maximum, remove components until back under, start with the smallest, smaller tasks are more likely to fit into an existing parent), rebalance quantities (1210) (e.g., set a parent to be equal to the total of its components with any overage and final adjustments, e.g., liquids to nearest 20 ml), and/or add detached components to existing flow (1210).

### Example Formula Management

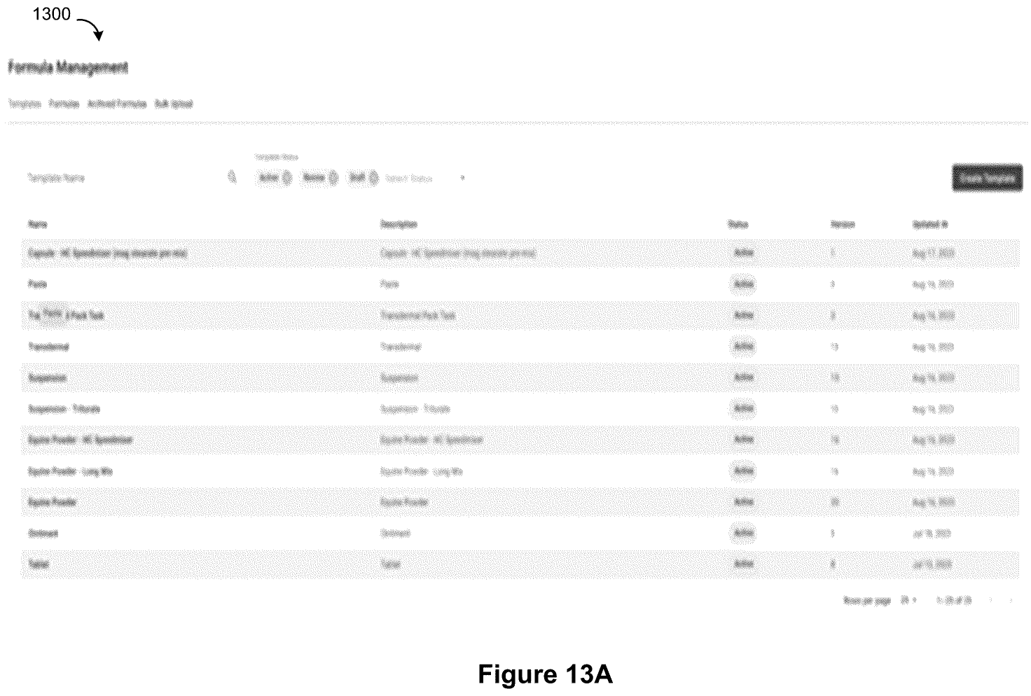

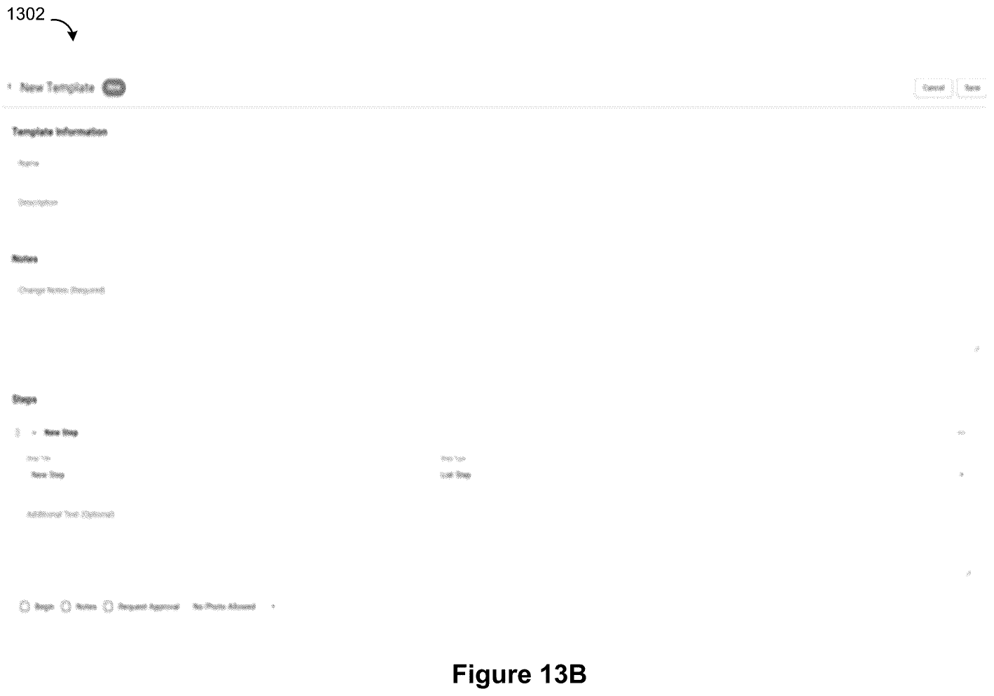

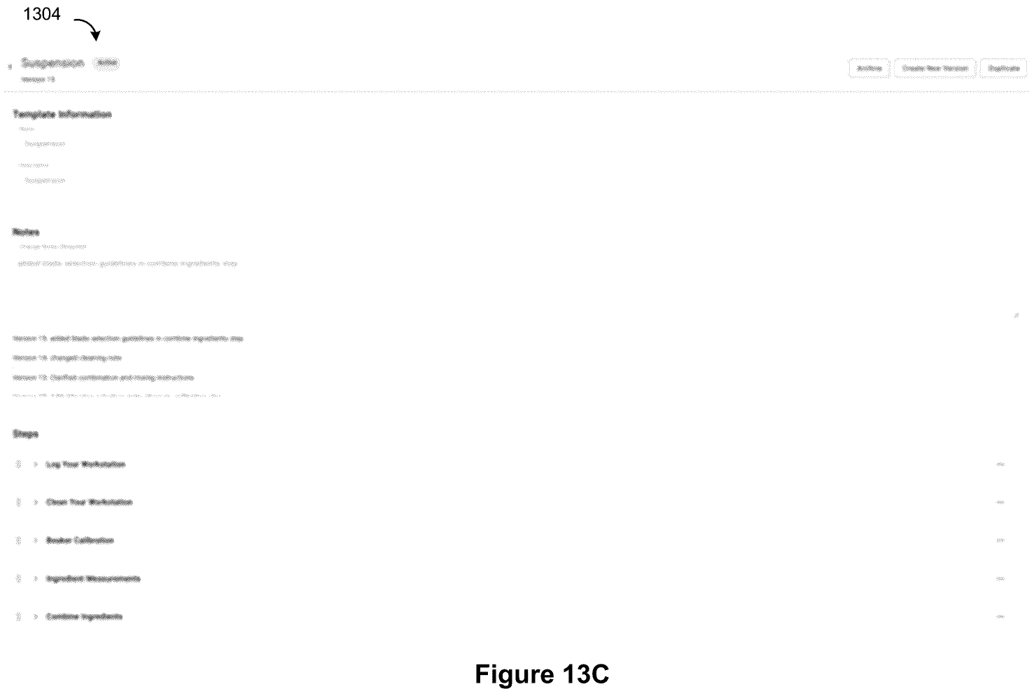

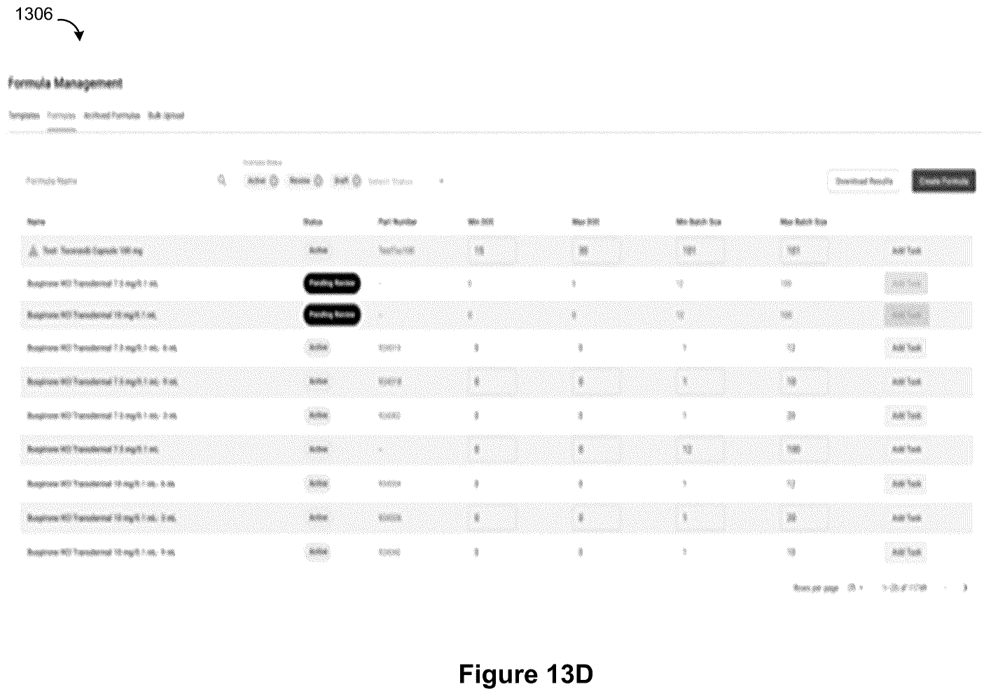

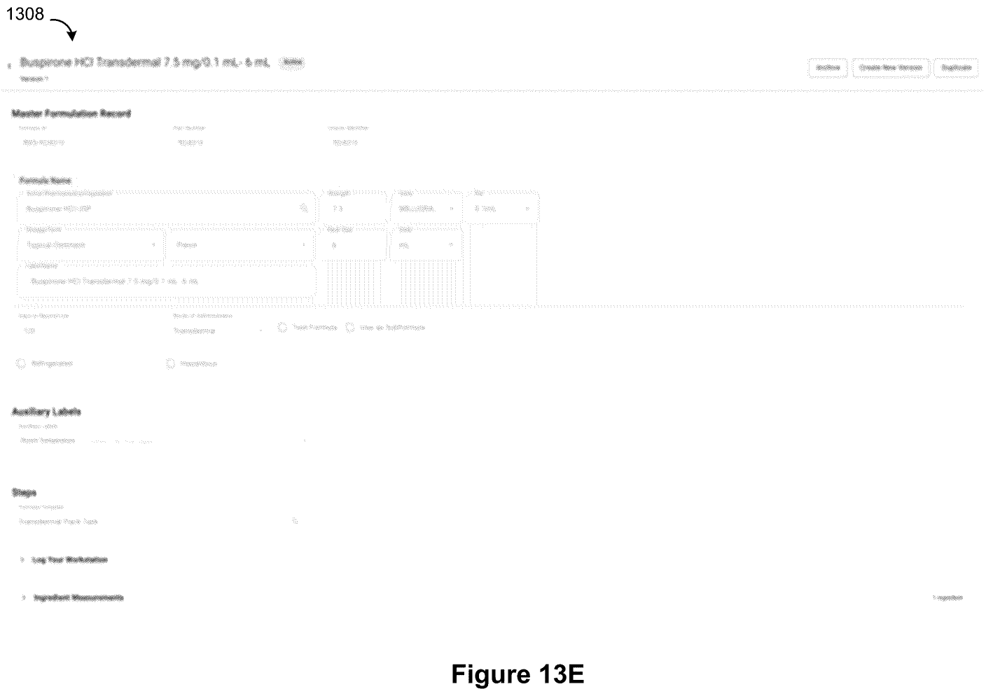

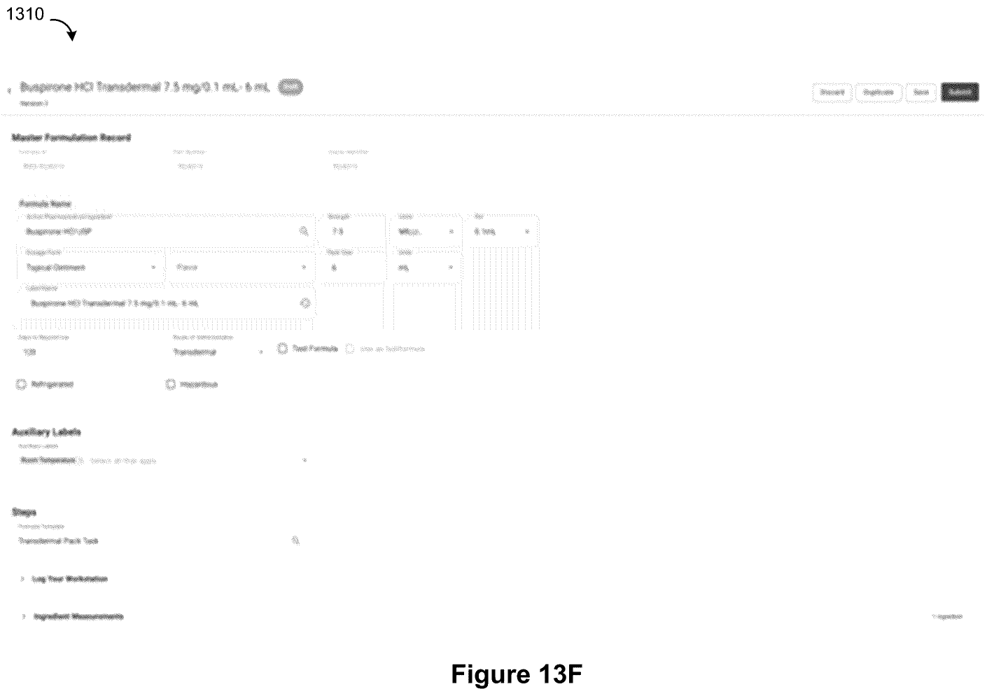

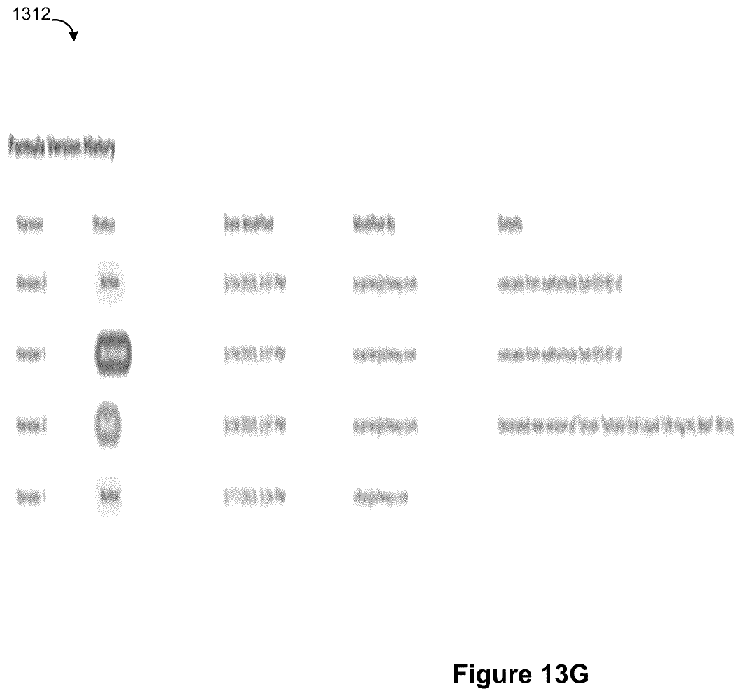

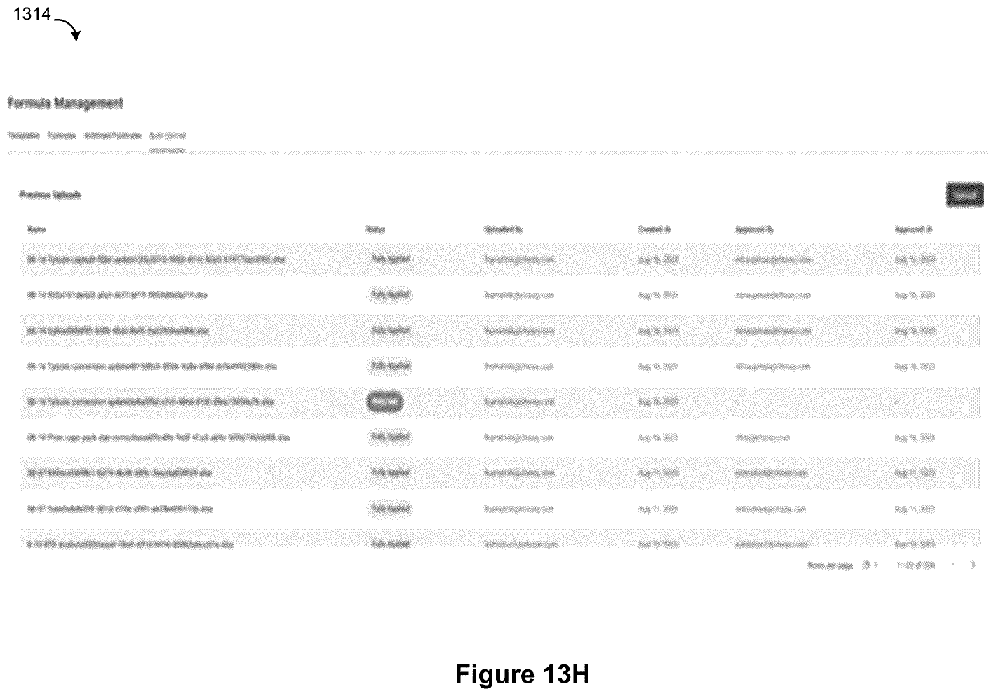

An example template landing page 1300 is shown in FIG. 13A, according to some embodiments. FIG. 13B shows an example template creation workflow 1302 when a pharmacist initiates a new version, according to some embodiments. FIG. 13C shows an example user interface 1304 for viewing and/or updating an existing template, according to some embodiments. FIG. 13D shows an example formula management landing page 1306, according to some embodiments. FIG. 13E shows an example user interface 1308 for viewing and/or updating an existing formula, according to some embodiments. FIG. 13F shows an example user interface 1310 for creating a newer version from an existing formula, according to some embodiments. FIG. 13G shows an example user interface 1312 for viewing version history, according to some embodiments. FIG. 13H shows an example user interface 1314 for bulk upload landing page, which may be used for performing uploads, according to some embodiments.

Example data elements in formula management are shown in the table below, according to some embodiments.

| Name | Description | Data Type |
|---|---|---|
| Formula id | same as SKU#: Oracle SKU# - RX5-###-## | Alpha |
| | | numeric |
| Chewy Part # | BO Part #, will be printed on stock bottles of the | Numeric |
| | finished compounds - At present it is stored as NDC | |
| Formula | This is system generated unique identifier. | ID |
| Template | | |
| API | Active Ingredient Label Name | Alpha |
| | | numeric |
| Dosage Form | formula dosage form (such as chews, oral liquids) | fixed set alpha |
| | | values |
| Strength | concentration of active API | decimal |
| Strength UOM | mg, mg/ml | fixed set of |
| | | values |
| Flavor | flavor to be included in finished product (such as | fixed set alpha |
| | chicken, marshmallow) | values |
| Pack Size | quantity per pack | Alpha |
| | | numeric |
| UoM Pack Size | Applicable Unit of measure per pack | fixed set of |
| | | values |
| Formula Name | Auto generated | alpha numeric |
| Days until Before | # in days to administer medication | numeric |
| use date (BUD) | | |
| Status | Formula Status - draft, pending review, active, inactive | fixed set alpha |
| | | values |
| Route of | Recommended route to administer medication (such as | fixed set alpha |
| administration | oral, topical) | values |
| Created by | capture username creating formula or template | alpha |
| Created DTTM | | Date Time |
| Updated by | capture username | alpha |
| Updated DTTM | | Date Time |
| Approved by | capture username | alpha |
| Approved DTTM | | Date Time |
| Total Quantity | Quantity that the formula is built for, this is important | Decimal |
| | in capturing the ratios. | |
| Quantity UOM | | |
| Total Weight | total weight for the total quantity - in mg | Decimal |
| Expected weight | expected weight of each in mg | Decimal |
| of each | | |
| Refrigerated Flag | if the product requires refrigeration (Yes or No) | Boolean |
| Hazardous Flag | if the product contains any hazardous material | Boolean |
| Aux Labels | Instructions or Warnings to be printed on a label. | fixed set alpha |
| | Note: a Formula can have more than 1 Aux Label: | values |
| | Example: “Shake well” , “Store at room temperature” | |
| Quantity | Quantity to be compounded in a batch | Decimal |
| Quantity Unit of | Quantity associated UoM (mg, mg/ml) | |
| Measure | | |
| Measurement | If flag is set to true, then when the compound is | Boolean |
| check | created, the pharmacist has to check and approve | |
| Quantity | Quantity Sufficient flag is used to have as filler to bring | Boolean |
| Sufficient | the overall volume to a specified level | |
| (QS)check | | |
| Monograph | This is a Notes field on the formula added from the | alpha-numeric |
| | supporting guidelines (such as US Pharma) | |
| Steps | This is derived from template in Formula as Step by | fixed set alpha |
| | step instruction to be performed in sequence. Example | values |
| | of Steps - Cleaning, Weighing, Pharmacist check 1, | |
| | Mixing, Pharmacist check 2, Moulding/Filling, | |
| | Pharmacist check Final. | |
| Step points | Each of the above tasks will have task points - measure | numeric |
| | of estimated time for each task - Defaulted from | |
| | Template - support editing task points | |
| Final Appearance | | |
| Packaging Note | | alphanumeric |
| Stability | Free text field | link |
| Supporting | | |
| Documentation | | |
| Reference | Free text field | link |
| Documentation | | |
| Storage | Free text field | |
| Instructions | | |
| Chemical | Free text field | |
| Compatibility | | |
| Version | When they need to make changes to an active formula, | System |
| | they put it to the proposed status which will be | generated |
| | assigned new version number, the old version must be | |
| | saved and retrievable when required | |
| Audit | Note: Audit for all the fields is only required to be | |
| | captured when the formula is in proposed /review | |
| | status. Status field should be audited in all the statuses. | |
| | Formula should not be editable in Active or Expired | |
| | status. Formula changes in new status is not required to | |
| | be audited. | |
| | Audit for MVP will only be of supported for Formula | |
| | Level attributes, any level 2, level 3 child attributes will | |
| | not be audited for MVP. | |
| | Changes to the level 2, level 3 child attributes will still | |
| | result in new version of the formula. | |
| Changed by | Username | alphanumeric |
| Changed Time | Date Time | Date Time |
| Attribute | Name of attribute that was changed | alphanumeric |
| Value before | | alphanumeric |
| change | | |
| Value after | | alphanumeric |
| change | | |

### Example Formula Management Life Cycle

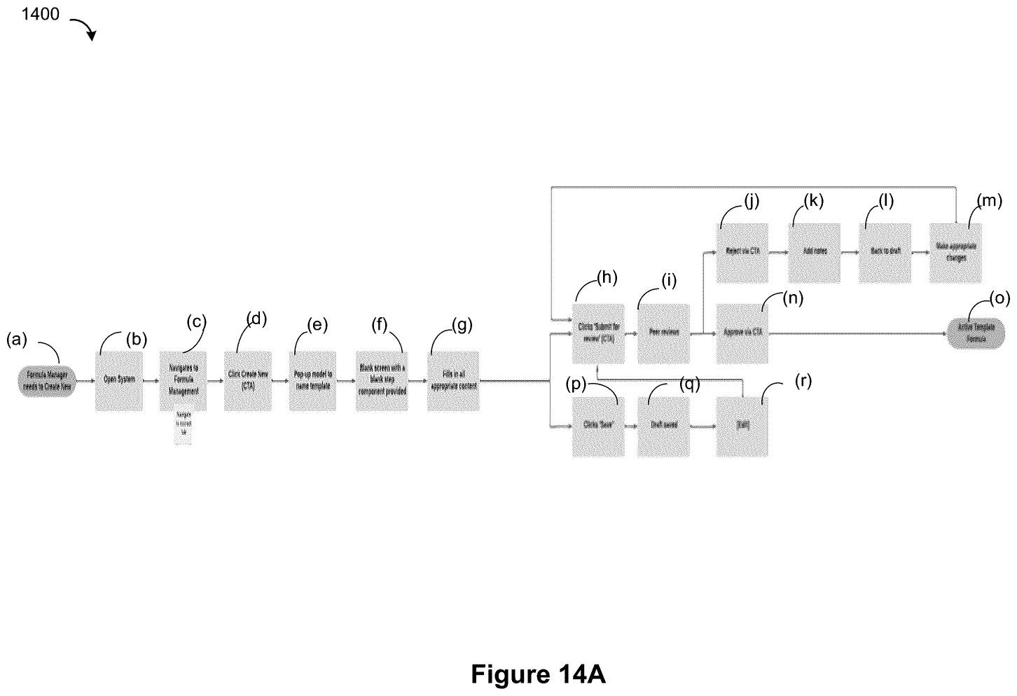

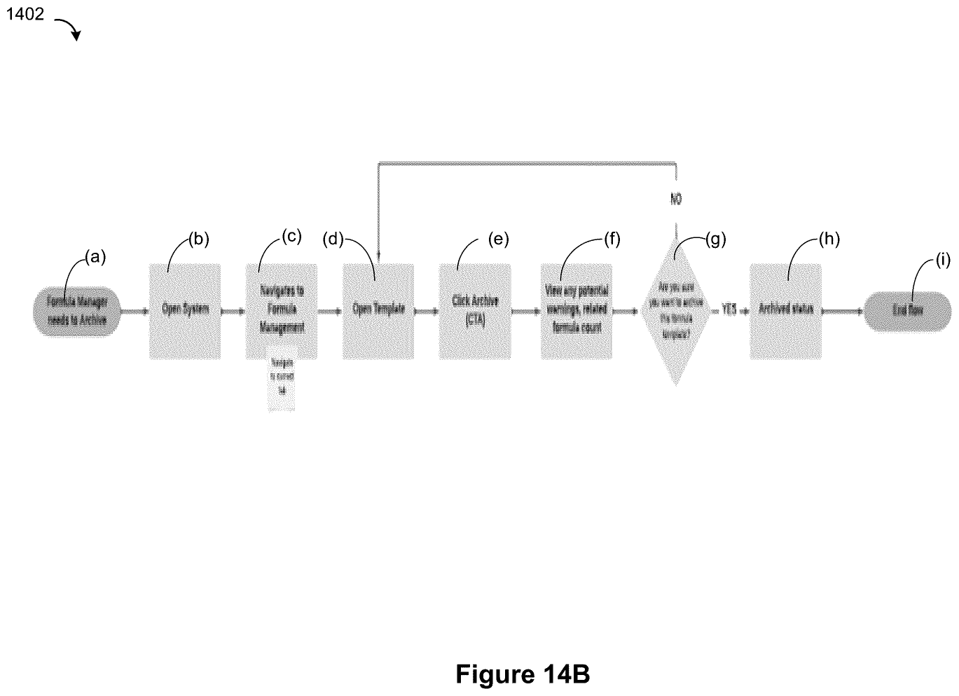

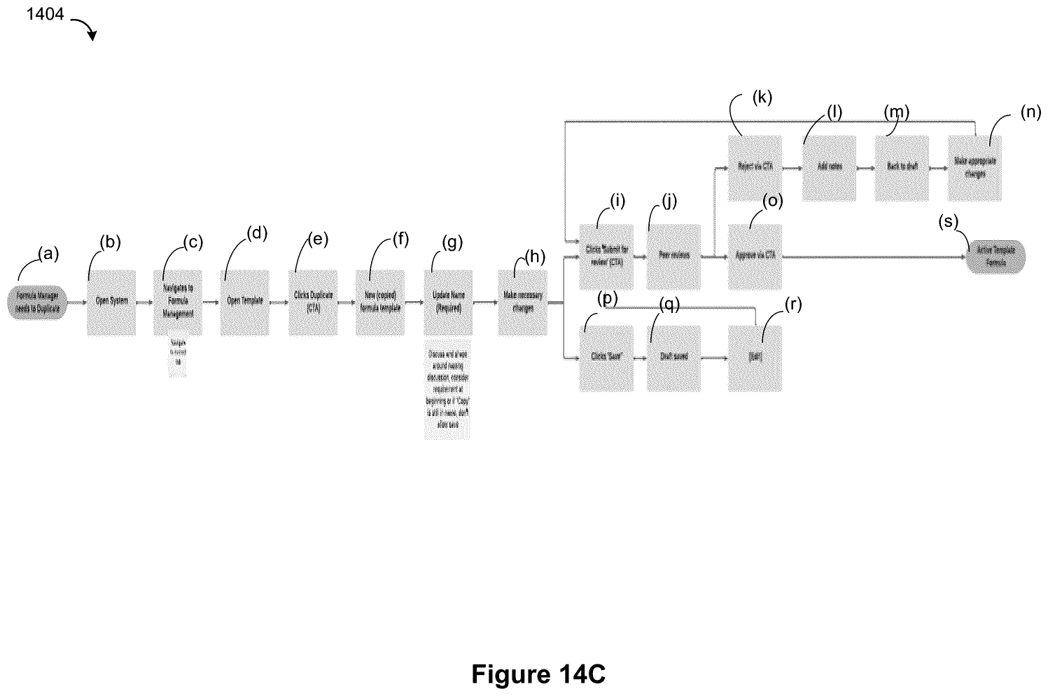

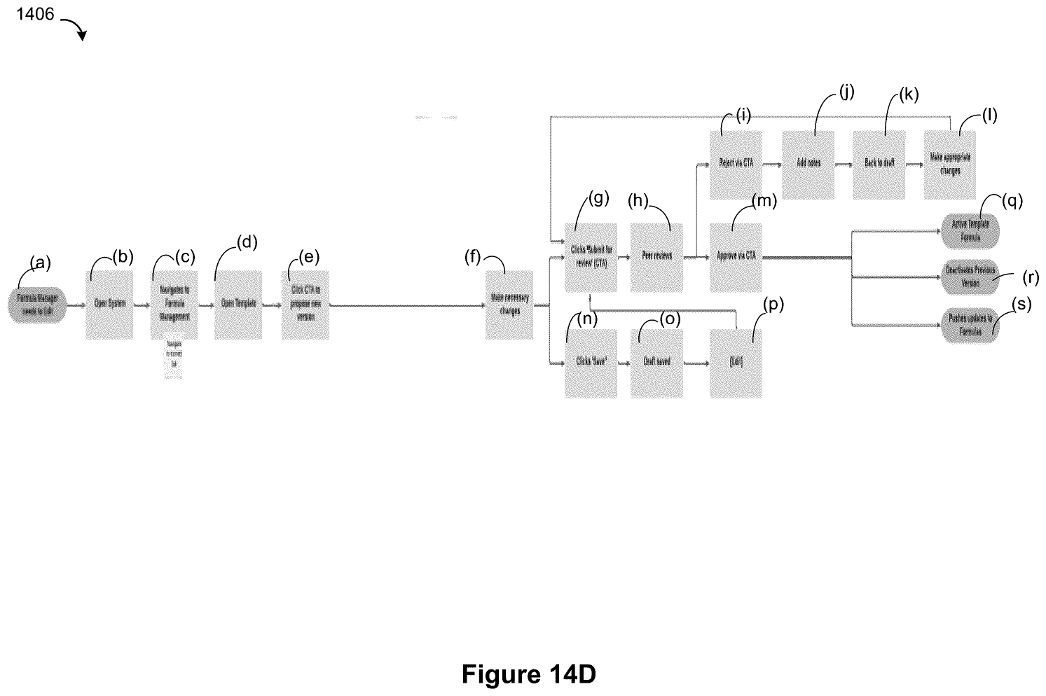

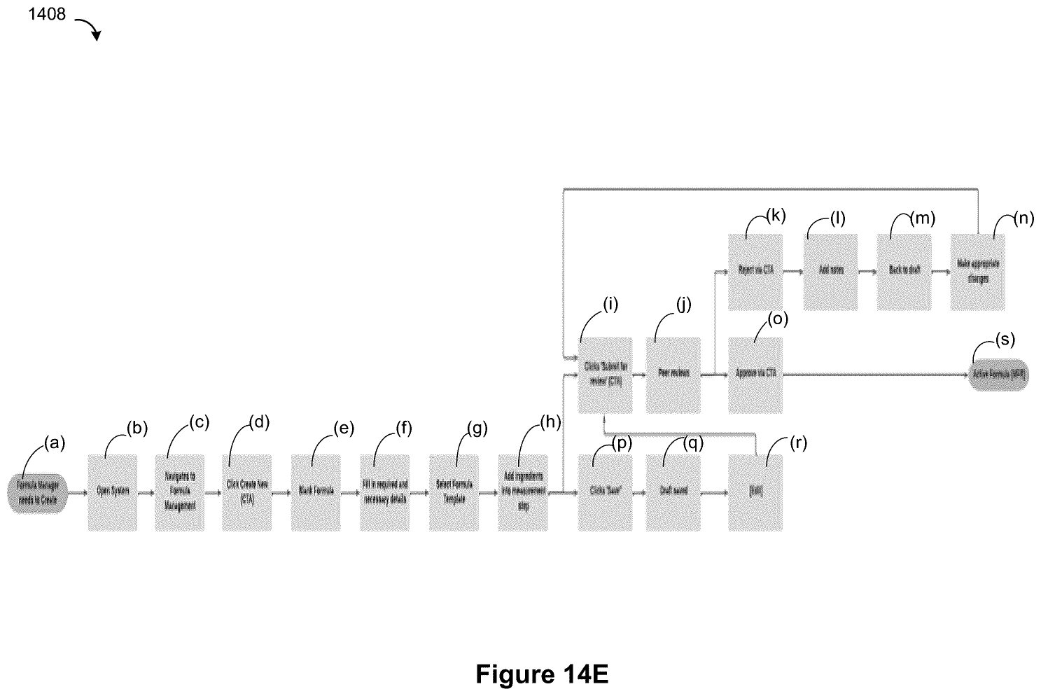

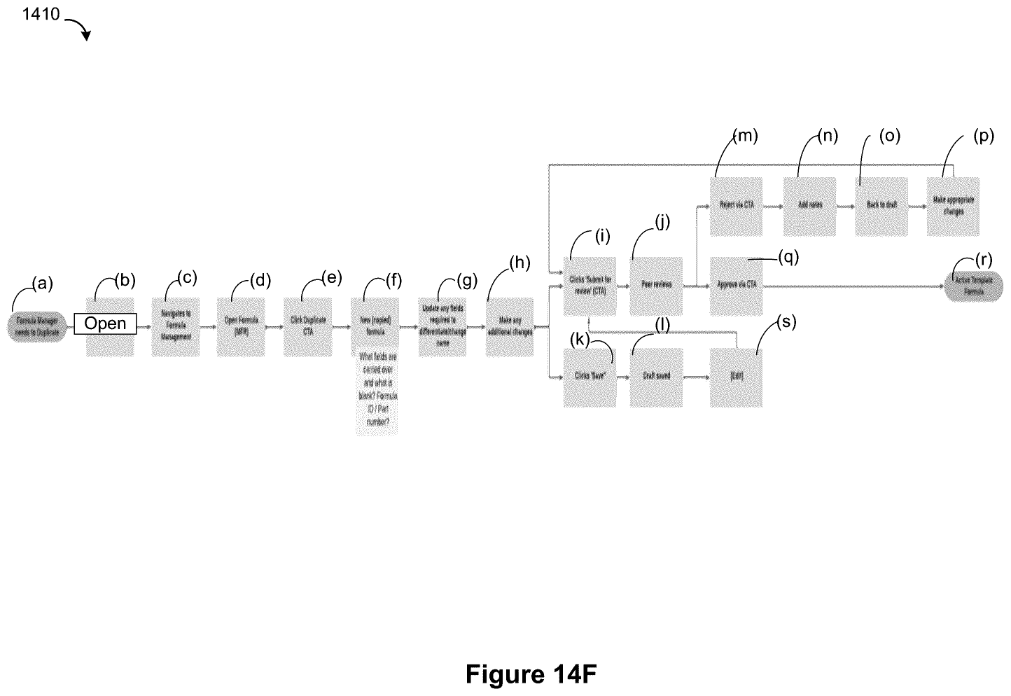

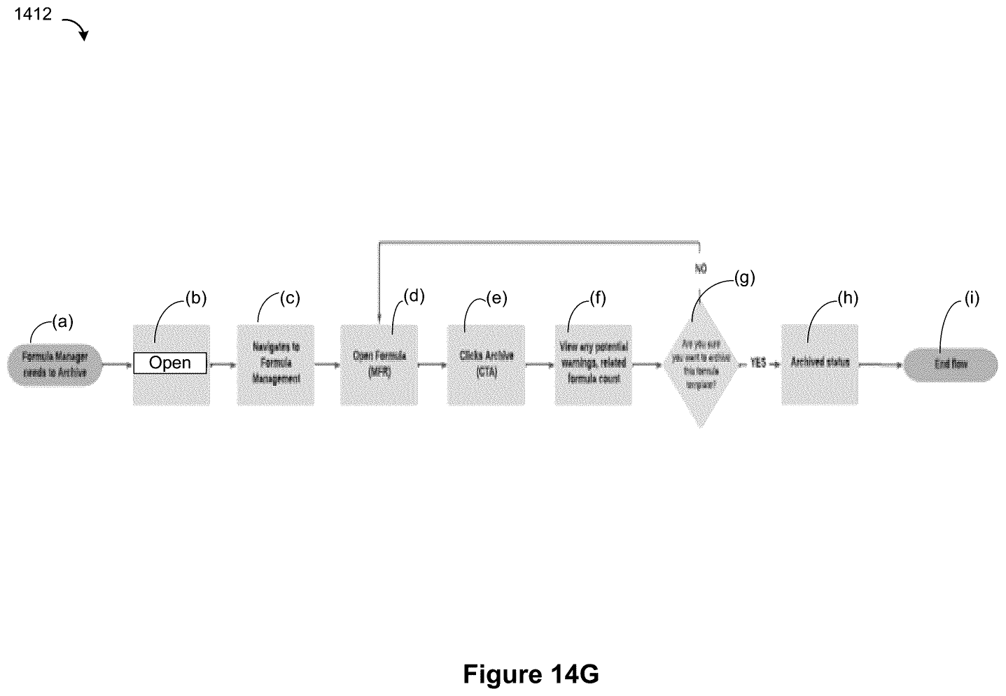

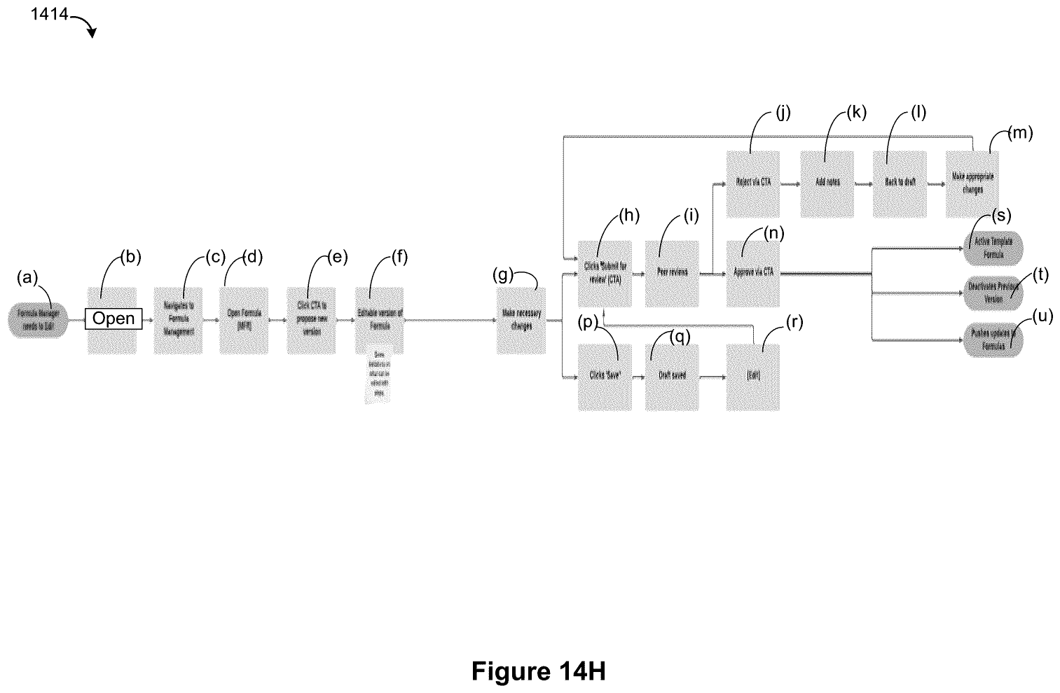

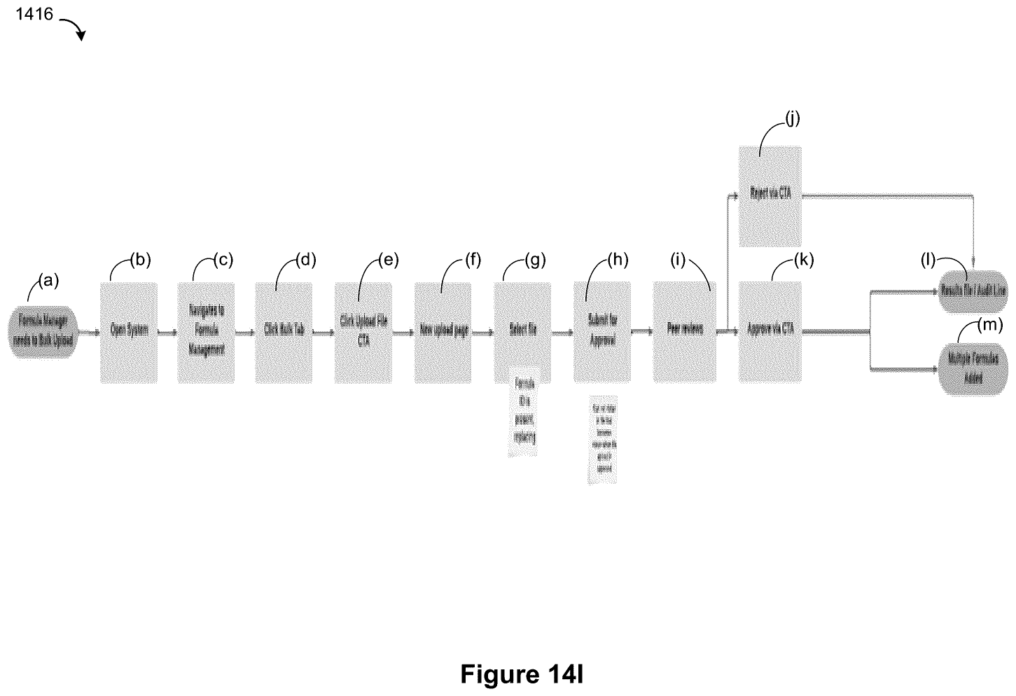

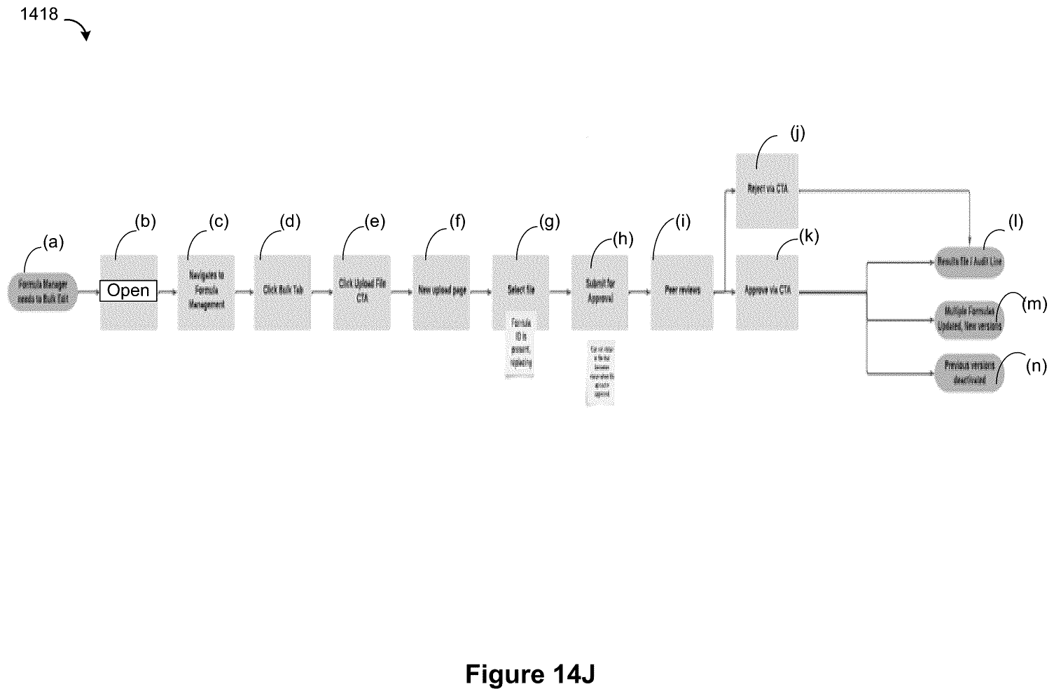

FIGS. 14A-14J show example flow diagrams for formula management, according to some embodiments. FIG. 14A shows an example flow 1400 for formula template creation. Suppose a formula manager needs to create (step (a)) a new template. The formula manager may open (step (b)) a system (e.g., a user interface connected to the system described herein) and may navigate (step (c)) to a formula management interface. The formula manager may then select or click (step (d)) create new template. The user interface may respond by popping up (step (e)) to name the template. A blank screen with a blank step component may be provided (step (f)). The formula manager may fill (step (g)) all appropriate content. The formula manager may then click (step (h)) a ‘submit for review’ and/or perform peer reviews (step (i)), either approve (step (n)) or reject (step (j)) the template. If the formula manager rejects the template, the formula manager may add notes (step (k)), go back to draft (step (l)), and/or make appropriate changes (step (m)). Approved templates may be designated as active template formula (step (o)). The formula manager may click ‘save’ (step (p)) upon which the draft may be saved (step (q)), and/or edited (step (r)), before the formula manager submits the template for review (step (h)).

FIG. 14B shows an example flow 1402 for formula template archiving. Suppose a formula manager needs to archive a template (step (a)). The formula manager may open (step (b)) a system or a relevant user interface, and/or navigate to a formula management (step (c)). The formula manager may open a template (step (d)), click an archive (step (e)), and/or view any potential warnings, related formula count (step (g)). The system or the user interface may prompt if the formula manager wants to archive the formula template (step (g)), and if so, set archived status (step (h)) and the flow may end (step (i)).

FIG. 14C shows an example flow 1404 for formula template duplication. Suppose a formula manager needs to duplicate (step (a)) a formula template. The formula manager may open (step (b)) a system or a relevant user interface, and/or navigate to a formula management (step (c)). The formula manager may open a template (step (d)), and/or click a duplicate (step (e)). Suppose the formula manager copies (step (f)) a formula template. The formula manager may update name (step (g)) and/or make necessary changes (step (h)). The formula manager may click ‘submit for review’ (step (i)), and/or peers may review (step (j)), approve (step (o)), in which case active template formula may be designated (step(s)). If peers reject (step (k)), the peers and/or the formula manager may add notes (step (l)), return to draft (step (m)), and/or make appropriate changes (step (n)) before returning to step (i). The formula manager may click ‘save’ (step (p)), in which case a draft may be saved (step (q)), before editing (step (r)), and returning to step (i).

FIG. 14D shows an example flow 1406 for formula template editing. Suppose a formula manager needs to edit (step (a)) a formula. The formula manager may open (step (b)) a system or a relevant user interface, and/or navigate to a formula management (step (c)). The formula manager may open a template (step (d)), and/or click to propose new version of the formula (step (e)). The formula manager may make necessary changes (step (f)) to a formula template. The formula manager may click ‘submit for review’ (step (g)), and/or peers may review (step (h)), and/or approve (step (m)), in which case active template formula may be designated (step (q)), previous version may be deactivated (step (r)), and/or updates may be pushed to formulas (step(s)). If peers reject (step (i)), the peers and/or the formula manager may add notes (step (j)), return to draft (step (k)), and/or make appropriate changes (step (l)) before returning to step (g). The formula manager may click ‘save’ (step (n)), in which case a draft may be saved (step (o)), before editing (step (p)), and returning to step (g).

FIG. 14E shows an example flow 1408 for formula creation. Suppose a formula manager needs to create (step (a)) a new formula. The formula manager may open (step (b)) a system (e.g., a user interface connected to the system described herein) and may navigate (step (c)) to a formula management interface. The formula manager may then select or click (step (d)) create new formula. The user interface may respond by showing a blank formula (step (e)) to fill the formula. The formula manager may fill in required and details (step (f)), select a formula template (step (g)), and/or add ingredients into measurement steps (step (h)). The formula manager may click ‘submit for review’ (step (i)), peers may review (step (j)), and/or approve (step (o)), in which case active formula may be designated (step(s)). If peers reject (step (k)), the peers and/or the formula manager may add notes (step (l)), return to draft (step (m)), and/or make appropriate changes (step (n)) before returning to step (i). The formula manager may click ‘save’ (step (p)), in which case a draft may be saved (step (q)), before editing (step (r)), and returning to step (i).

FIG. 14F shows an example flow 1410 for formula duplication. Suppose a formula manager needs to duplicate (step (a)) a formula. The formula manager may open (step (b)) a system or a relevant user interface, and/or navigate to a formula management (step (c)). The formula manager may open a formula (step (d)), and/or click a duplicate (step (e)). Suppose the formula manager copies (step (f)) a formula. The formula manager may update any fields required to differentiate the formula from other formula and/or change name (step (g)) and/or make any additional changes (step (h)). The formula manager may click ‘submit for review’ (step (i)), and/or peers may review (step (j)), approve (step (q)), in which case active template formula may be designated (step (r)). If peers reject (step (m)), the peers and/or the formula manager may add notes (step (n)), return to draft (step (o)), and/or make appropriate changes (step (p)) before returning to step (i). The formula manager may click ‘save’ (step (k)), in which case a draft may be saved (step (l)), before editing (step(s)), and returning to step (i).

FIG. 14G shows an example flow 1412 for formula archiving. Suppose a formula manager needs to archive a formula (step (a)). The formula manager may open (step (b)) a system or a relevant user interface, and/or navigate to a formula management (step (c)). The formula manager may open a formula (step (d)), click an archive (step (e)), and/or view any potential warnings, related formula count (step (f)). The system or the user interface may prompt if the formula manager wants to archive the formula (step (g)), and if so, set archived status (step (h)) and the flow may end (step (i)).

FIG. 14H shows an example flow 1414 for formula editing. Suppose a formula manager needs to edit (step (a)) a formula. The formula manager may open (step (b)) a system or a relevant user interface, and/or navigate to a formula management (step (c)). The formula manager may open a formula (step (d)), and/or click to propose new version of the formula (step (e)). An editable version of formula may be shown (step (f)). The formula manager may make necessary changes (step (g)) to the formula. The formula manager may click ‘submit for review’ (step (h)), and/or peers may review (step (i)), and/or approve (step (n)), in which case active formula may be designated (step(s)), previous version may be deactivated (step (t)), and/or updates may be pushed to formulas (step (u)). If peers reject (step (j)), the peers and/or the formula manager may add notes (step (k)), return to draft (step (l)), and/or make appropriate changes (step (m)) before returning to step (h). The formula manager may click ‘save’ (step (p)), in which case a draft may be saved (step (q)), before editing (step (r)), and returning to step (h).

FIG. 14I shows an example flow 1416 for formula bulk upload. Suppose a formula manager needs to bulk upload formula (step (a)). The formula manager may open (step (b)) a system or a relevant user interface, and/or navigate to a formula management (step (c)). The formula manager may click bulk tab (step (d)), and/or click upload file (step (e)). The system or the user interface may show a new upload page (step (f)). The formula manager may select a file (step (g)), submit for approval (step (h)), and/or peer reviews (step (i)). If approved (step (k)), result file or audit line may be created (step (l)), and/or multiple formulas may be added (step (m)). If rejected (step (j)), only the result file or audit line may be added/created (step (l)).

FIG. 14J shows an example flow 1418 for formula bulk edit. Suppose a formula manager needs to bulk edit formula (step (a)). The formula manager may open (step (b)) a system or a relevant user interface, and/or navigate to a formula management (step (c)). The formula manager may click bulk tab (step (d)), and/or click upload file (step (e)). The system or the user interface may show a new upload page (step (f)). The formula manager may select a file (step (g)), submit for approval (step (h)), and/or peer reviews (step (i)). If approved (step (k)), result file or audit line may be created (step (l)), and/or multiple formulas may be updated (step (m)), and/or previous versions may be deactivated (step (n)). If rejected (step (j)), only the result file or audit line may be added/created (step (l)).

### Example Ingredient Management Flows

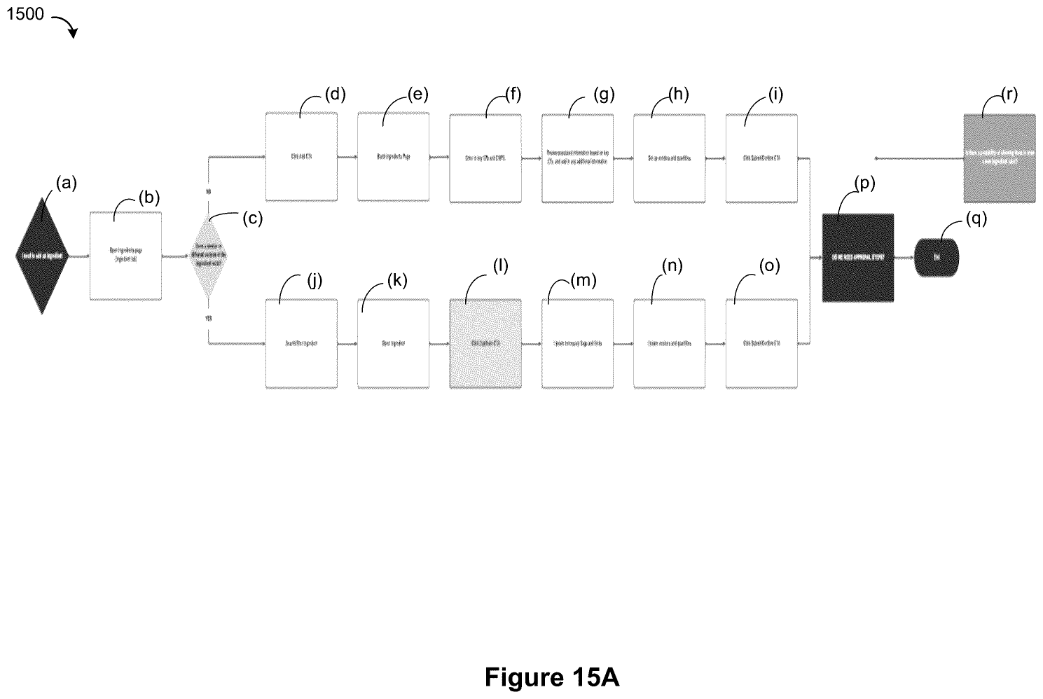

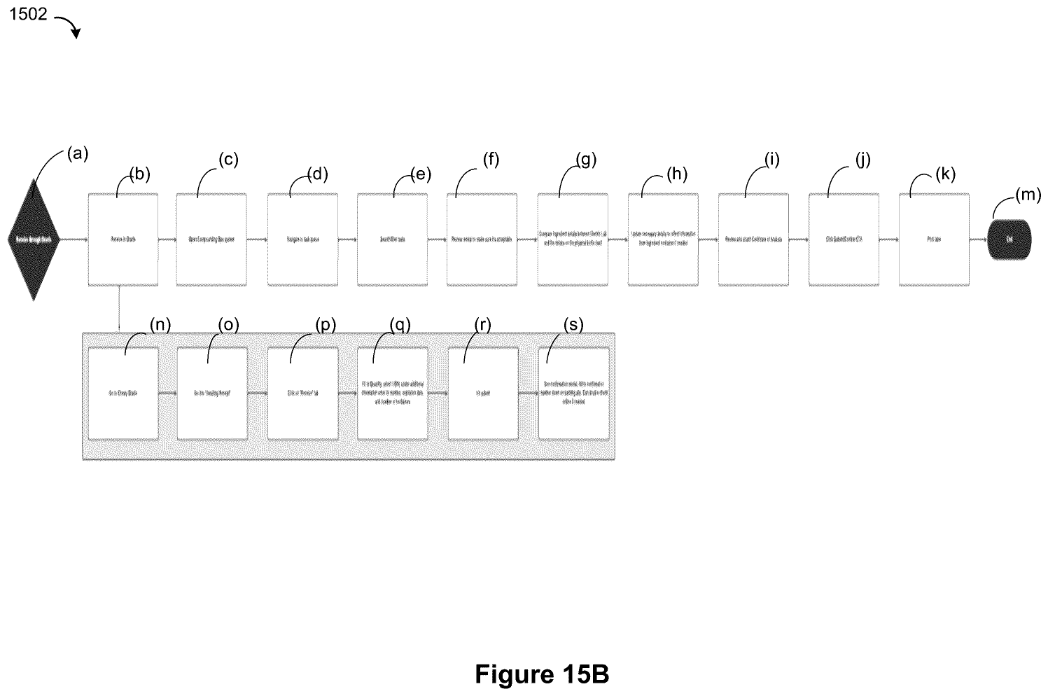

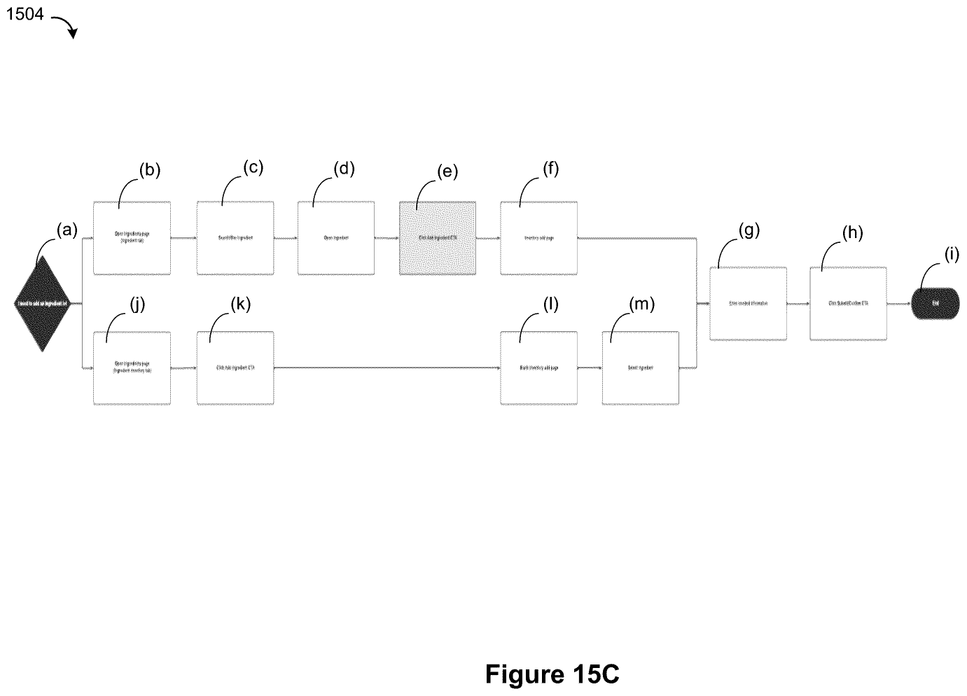

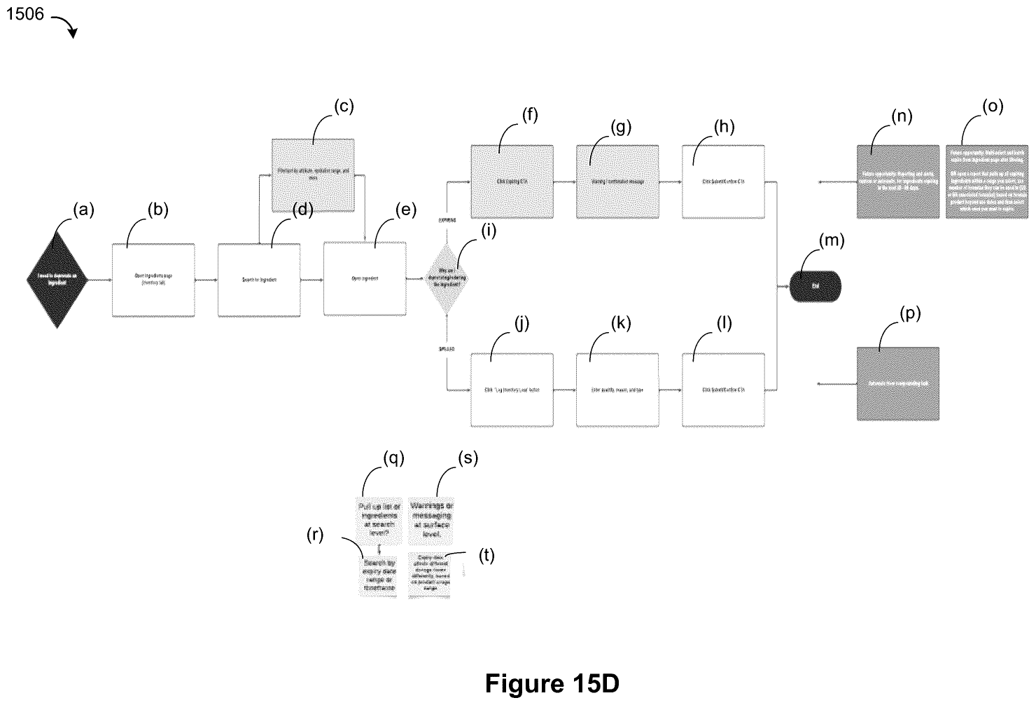

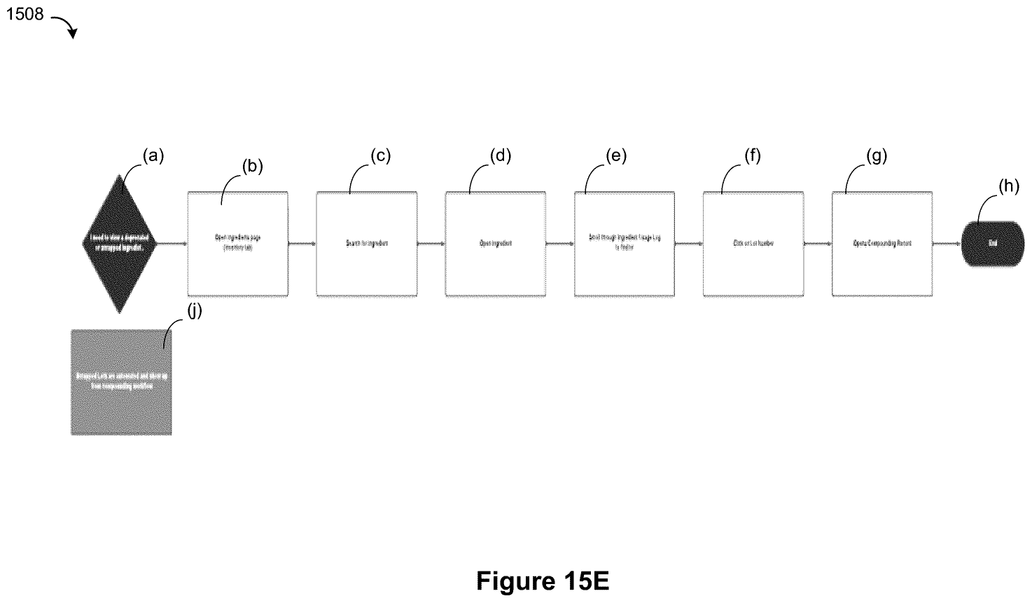

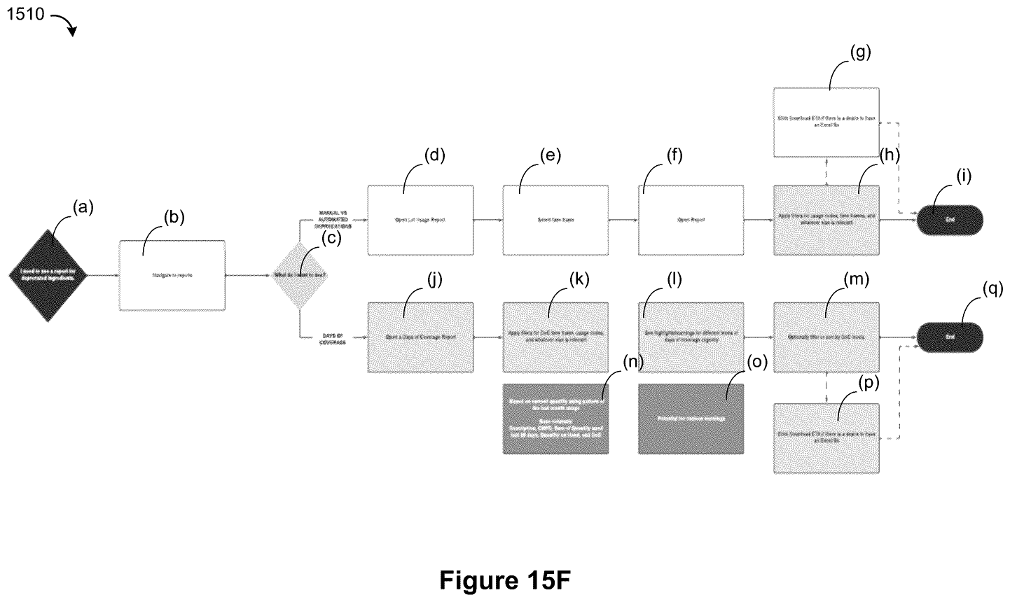

FIGS. 15A-15F show example flow diagrams for ingredient management, according to some embodiments. FIG. 15A shows an example flow 1500 for adding an ingredient. Suppose a pharmacist needs to add an ingredient (step (a)), the pharmacist may open an ingredients page or an ingredient tab (step (b)). The pharmacist may view to determine if a similar or different version of the ingredient exists (step (c)). If the ingredient does not exist, the pharmacist may click add (step (d)), a blank ingredients page may be shown (step (e)), following which the pharmacist may enter in key identifier(s) (step (f)), review populated information based on key identifiers and add in any additional information (step (g)), set up vendors and quantities (step (h)), and/or click submit or confirm (step (i)). If the ingredient does exist, the pharmacist may search or filter ingredient (step (j)), open the ingredient (step (k)), click duplicate (step (l)), update necessary flags and fields (step (m)), update vendors and quantities (step (n)), and/or click submit or confirm (step (o)). If there are no approval steps needed ((step (p)), the process may end (step (q)). The system may allow the pharmacist to scan a new ingredient label (step (r)).

FIG. 15B shows an example flow 1502 when a new ingredient arrives at the system (e.g., via a relational database). Suppose an ingredient is received through a relational database, such as Oracle (step (a)). The system may receive (step (a)) the ingredient automatically (e.g., by making an entry to a table of the database and/or a pharmacist marking the ingredient as received. The pharmacist may open a compounding operations system (step (c)), navigate to a task queue (step (d)), search or filter tasks (step (e)), review receipt to make sure it is acceptable (step (f)), compare ingredient details between the system and the details on a physical bottle (step (g)), update necessary details to reflect information from ingredient container if needed (step (h)), review and attach any certificates (step (i)), click submit or confirm (step (j)), and/or print label (step (k)), before the process ends (step (m)). The pharmacist may navigate to a business specific database (step (n)) and an awaiting receipt (step (o)), click on a receiver tab (step (p)), fill in quantity, lot number, expiration data, and number of containers (step (q)), hit submit (step (r)), and/or see confirmation modal, write confirmation number down on packing slip, and/or double check online as needed (step(s)).

FIG. 15C shows an example flow 1504 when a pharmacist needs to add an ingredient lot (step (a)). The pharmacist may open an ingredient page and/or an ingredient tab (step (b)). The pharmacist may search/filter ingredient (step (c)), open the ingredient (step (d)), click an add ingredient (step (e)), and/or navigate to an inventory add page (step (f)). Alternatively, the pharmacist may open an ingredients page (step (j)), click an add ingredient (step (k)), navigate to a blank inventory add page (step (l)), and/or select an ingredient (step (m)). The flow may also include entering needed information (step (g)), clicking submitting (step (h)), prior to ending (step (i)).

FIG. 15D shows an example flow 1506 when a pharmacist needs to deprecate an ingredient (step (a)). The pharmacist may open an ingredients page or an inventory tab FIG. 15C shows an example flow 1504 when a pharmacist needs to add an ingredient lot (step (b)), search for an ingredient (step (d)), (optionally) filter or sort by attribute, expiration range (step (c)), and/or open an ingredient (step (e)). The pharmacist may choose one of two paths based on whether the pharmacist is deprecating or reducing the ingredient (step (i)). If it is for expired ingredients, the pharmacist may click expiring (step (f)), after which a warning confirmation page may be shown (step (g)), and/or the pharmacist may click submit or confirm (step (h)). If it is for spilled ingredients, the pharmacist may click ‘log inventory loss’ button (step (j)), enter quantity, reason, and type (step (k)), and/or click submit or confirm (step (l)). Some embodiments report and/or alert, (step (n)) (custom or automatic) for ingredient expiring in a next time period (e.g., in the next 30 to 60 days). Some embodiments provide multi-select and batch expire from ingredient batch after filtering, and/or open a report that pulls up expiring ingredients within a range the pharmacist selects. Some embodiments may allow the pharmacist to see number of formulas they can be used in based on formula product beyond use dates and then select which ones the pharmacist wants to expire (step (o)). Some embodiments automate from compounding task (step (p)). Some embodiments pull up a list of ingredients at search level (step (q)), and/or allow the pharmacist to search by expiry date range or timeframe (step (r)). Some embodiments provide warnings or messaging at surface level (step(s)). In some embodiments, expiry date affects different dosage forms differently, based on product usage range (step (t)). The process may end after one or more steps (step (m)) described above.

FIG. 15E shows an example flow 1508 when a pharmacist needs to view a deprecated or scrapped ingredient (step (a)). The pharmacist may open an ingredients page (step (b)), search for an ingredient (step (c)), open an ingredient (step (d)), scroll through an ingredient usage log to find a lot (step (e)), click on a lot number (step (f)), and/or open compounding record (step (g)), after which the process may end (step (h)). Scrapped lots may be automated and/or may show up from compounding workflow (step (j)).

FIG. 15F shows an example flow 1510 when a pharmacist needs to see a report for deprecated ingredients (step (a)). The pharmacist may navigate to reports (step (b)). The pharmacist may choose one of two flows depending on what they want to see (step (c)). If the pharmacist wants to see manual or automated deprecations, the pharmacist may open a lot usage report (step (d)), select a time frame (step (e)), open a report (step (f)), apply filters for usage codes, time frames, any relevant data (step (h)), optionally click download if a spreadsheet is needed (step (g)), after which the process may end (step (j)). If the pharmacist wants to see days of coverage, the pharmacist may open a days of coverage report (step (j)), apply filters for DoC time frame, usage codes and relevant data (step (k)), see highlights/warning for different levels of days of coverage urgency (step (l)), optionally filter or sort by DoC levels (step (m)), optionally click download if a spreadsheet is needed (step (p)), after which the process may end (step (q)). In some embodiments, the filters may be applied based on current quantity using pattern of the last month usage, and base columns may include description, sum of quantity used in last 30 days, quantity on hand, and/or DoC (step (n)). Some embodiments may provide warnings customized by the pharmacist and/or other users (step (o)).

### Example Devices

A scale connector (e.g., the scale connector 14, FIG. 3) may be connected to one or more devices, such as a scale. A scale may be discovered as follows. To create a scale, a physical scale may be setup. These may be configured in person. A user interface may show a menu with communication, Ethernet, device settings, input internet protocol (IP) address, port number, default gateway, and/or subnet mask. Other example menu options may include communication, Ethernet, print settings, data transfer function (which be activated to “On”, interval set to 1 seconds, for e.g.,). A scale may be registered in the ops portal via a “Create Scale” form on a scale management page. Physical scales may require an IP and/or port that line up with the previously entered values. Scales may only accept one connection at a time. If the same IP is registered in both staging and production, connection issues may occur. When a scale is created and enabled, the scale may be picked up by a scale gateway in an Internet-of-Things (IoT) Gateway. Scales may be picked up by a scale gateway in a round robin algorithm. If there are issues with a current scale or gateway combo, reassignment may be triggered manually through a “reconnect scale” button on the scale management page. A scale's reading may be inserted into a scale measurement table (e.g., every second) from an IoT gateway data service. A weight step may only accept readings if they are under a certain latency.

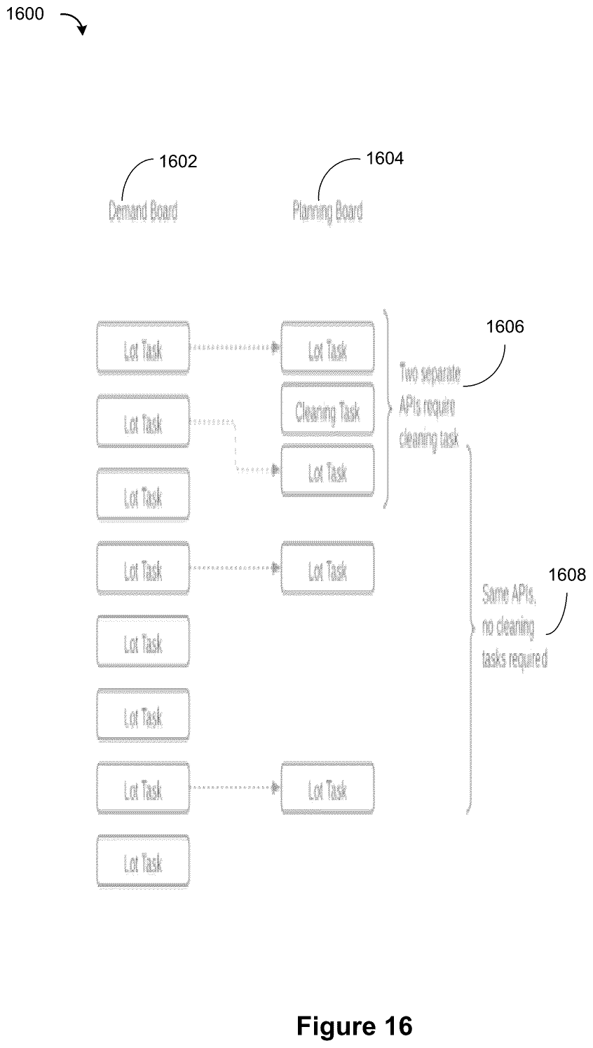

FIG. 16 is a schematic diagram for example demand and planning boards 1600, according to some embodiments. Lot tasks for a demand board 1602 (e.g., a landing page, a laundry list of jobs) and lot tasks for a planning board 1604 (e.g., a board that a pharmacist focuses on) are shown. Lot tasks in the demand board may be mapped to lot tasks and/or cleaning tasks in the planning board. Two separate APIs 1606 may require cleaning task. For some lot tasks 1608, same APIs may be used and/or no cleaning steps may be needed. Cleaning step may be used to define containers to use for compounding, tasks having cleaning agents. Cleaning step may be different depending on different dosage forms and tasks, chemicals to be used, etc. A workflow for the demand board may include a pharmacist looking at a highest priority (or higher priority than other) unassigned lot tasks. Another workflow may include a pharmacist filtering demand board by lot tasks with the same API and/or dosage form. Another workflow may include a pharmacist assigning a predetermined number (e.g., 3) lot tasks to a workstation and/or technician, which may implicitly set a priority to urgent. The planning board may show a current plan (e.g., for a day) based on time estimates to complete all tasks, including cleaning. Re-assignment may also happen on this board. The planning board may show both completed and incomplete lot tasks.

### Examples Methods for Automating Compounding Pharmacies

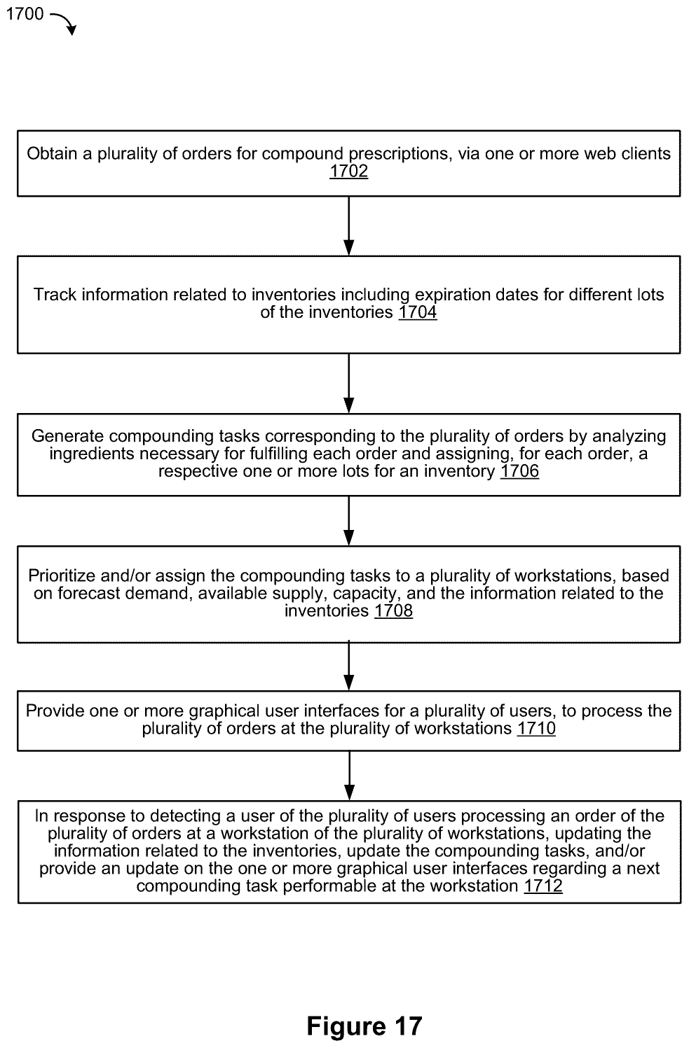

FIG. 17 shows a flow chart of an example method 1700, according to some embodiments. The method 1700 may be performed by the system 100 described above in reference to FIG. 1, according to some embodiments. The method may be used for automating preparation of compound prescription at a plurality of workstations having integrated therein a user interface and a registered quantity input hardware, based on forecasted quantities, ordered quantities and simultaneous usage data.

The method may include obtaining (1702) a plurality of orders for compound prescriptions, via one or more web clients. The method may also include tracking (1704) information related to inventories including expiration dates for different lots of the inventories. The method may also include generating (1706) compounding tasks corresponding to the plurality of orders by analyzing ingredients necessary for fulfilling each order and assigning, for each order, a respective one or more lots for an inventory. The method may also include prioritizing and assigning (1708) the compounding tasks to a plurality of workstations, based on forecast demand, available supply, capacity, and the information related to the inventories. The method may also include providing (1710) one or more graphical user interfaces for a plurality of users, to process the plurality of orders at the plurality of workstations. The method may also include in response to detecting a user of the plurality of users processing an order of the plurality of orders at a workstation of the plurality of workstations, updating (1712) the information related to the inventories, updating the compounding tasks, and/or providing an update on the one or more graphical user interfaces regarding a next compounding task performable at the workstation.

In some embodiments, the method may include interfacing with at least one device selected from the group consisting of: one or more scales, one or more scanners, and one or more mixers, and detecting a status of the order of the plurality of orders at the workstation, by obtaining one or more signals from the at least one device.

In some embodiments, the method may further include retrieving one or more templates corresponding to the plurality of orders, wherein each template includes a set of instructions for a workflow and a formula, and providing, via the one or more graphical user interfaces, details to implement the formula using the workflow, at the plurality of workstations.

In some embodiments, the one or more templates are customizable based upon one or more devices connected to each workstation.

In some embodiments, the method may include providing to a first set of users of the plurality of users a dashboard to update the one or more templates, and upon receiving an update the one or more templates, providing the updates, via the one or more graphical user interfaces.

In some embodiments, the capacity may correspond to at least one of a number of workstations, and a number of users.

In some embodiments, the available supply may be tracked based on tracking information related to the inventories, at one or more warehouses, and/or at the plurality of workstations. An inventory service may provide information on inventory. Such information may include the level of inventory, warehouses that have the inventory, and the level of inventory at each facility.

In some embodiments, the forecast demand may be obtained based on a forecasting model that is trained to predict demand based on historical data for compound prescription from the one or more web clients. The forecasting model may be trained using daily forecast from an operations team. Such forecast may be available at location level. Forecast may be for an SKU, and/or for different locations. Forecast may be based on every part number, e.g., at SKU level.

In some embodiments, prioritizing and assigning the compounding tasks to the plurality of workstations may be based on planned shifts of the plurality of users, and/or operational efficiency of the plurality of workstations.

In some embodiments, prioritizing and assigning the compounding tasks to the plurality of workstations may be based on forecasting, inventory, outstanding orders, a time an order was approved, and target days of coverage.

In some embodiments, the method may further include providing, via the one or more graphical user interfaces, for the user, while the user is processing the order, a real-time update regarding a weight of an ingredient and an expected range of weight for the ingredient according to a formula. The method may also include in response to detecting the weight of the ingredient is stabilized, displaying a final weight of the ingredient, allowing the user to record, and/or automatically recording the final weight of the ingredient for the order.

In some embodiments, the method may further include providing, via the one or more graphical user interfaces, for a super user different from the user processing the order, after the user processes the order, a summary of the processing of the order. The method may also include in response to receiving a negative input from the super user regarding an incorrect compounding for the order, requeuing a task corresponding to the order to the plurality of workstations.

In some embodiments, prioritizing and assigning the compounding tasks to the plurality of workstations may be based on batching one or more tasks that use an ingredient.

In some embodiments, prioritizing and assigning the compounding tasks to the plurality of workstations may be based on ordering tasks for one or more customers responsible for the plurality of orders received via the one or more web clients.

In some embodiments, prioritizing and assigning the compounding tasks to the plurality of workstations may be based on a demand for one or more ingredients.

In some embodiments, the method may include prioritizing and assigning the compounding tasks to the plurality of workstations may be based on task initiation prompted by a user request through the one or more graphical user interfaces thereby providing on-demand control.

In some embodiments, the method may include prioritizing and assigning the compounding tasks to the plurality of workstations may be automatically triggered through a daemon to execute scheduled commands job.

In some embodiments, each compound may include a corresponding minimum inventory threshold to trigger a new order and/or a minimum batch size for a next order.

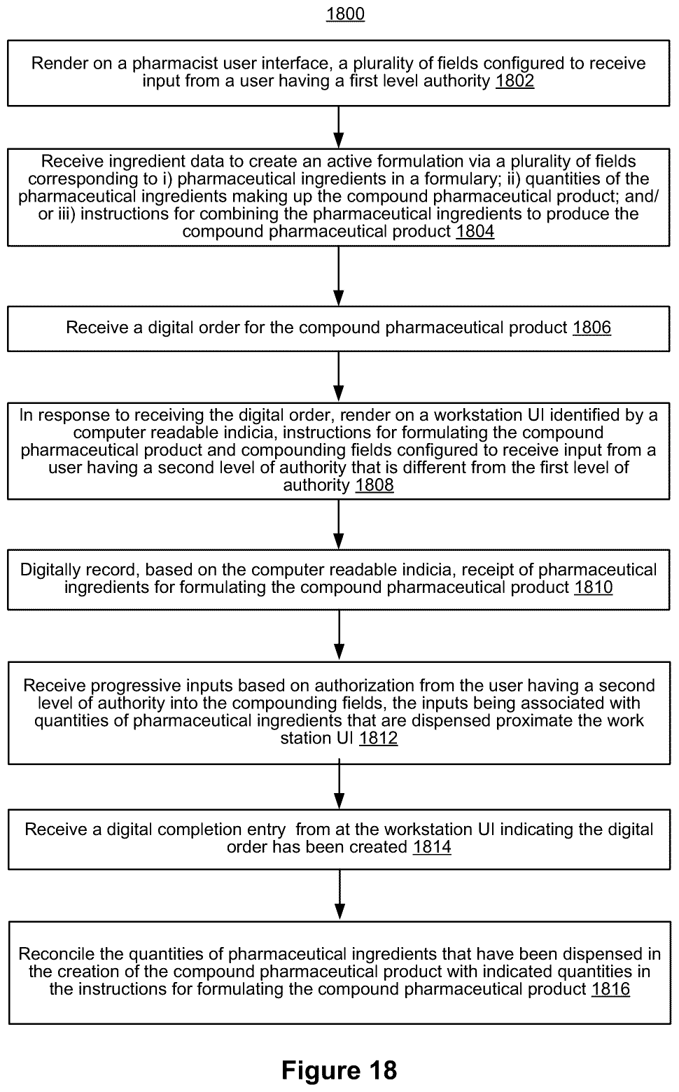

FIG. 18 shows a flow chart of an example method 1800, according to some embodiments. The method 1800 may be performed by the system 100 described above in reference to FIG. 1, according to some embodiments. The method may be used for guiding the fulfillment of a compound pharmaceutical product. The method may include rendering (1802) on a pharmacist user interface, a plurality of fields configured to receive input from a user having a first level authority. The method may also include receiving (1804) ingredient data to create an active formulation via a plurality of fields corresponding to i) pharmaceutical ingredients in a formulary; ii) quantities of the pharmaceutical ingredients making up the compound pharmaceutical product; iii) instructions for combining the pharmaceutical ingredients to produce the compound pharmaceutical product. The method may also include receiving (1806) a digital order for the compound pharmaceutical product. In response to receiving the digital order, the method may also include rendering (1808) on a workstation UI identified by a computer readable indicia, instructions for formulating the compound pharmaceutical product and compounding fields configured to receive input from a user having a second level of authority that is different from the first level of authority. The method may also include digitally recording (1810), based on the computer readable indicia, receipt of pharmaceutical ingredients for formulating the compound pharmaceutical product. The method may also include receiving (1812) progressive inputs based on authorization from the user having a second level of authority into the compounding fields, the inputs being associated with quantities of pharmaceutical ingredients that are dispensed proximate the workstation UI. The method may also include receiving (1814) a digital completion entry from at the workstation UI indicating the digital order has been created. The method may also include reconciling (1816) the quantities of pharmaceutical ingredients that have been dispensed in the creation of the compound pharmaceutical product with indicated quantities in the instructions for formulating the compound pharmaceutical product.

Formula task may not be specific for each equipment. A formula task may specify that a specific homogenizer may need to be used for a specific task, and/or a specific equipment may be used for each task. An example of different steps that may be taken for a single order, different dosage forms that are being used, and/or different unit of measures, may be listed in a formula. The customizability makes the system more scalable. Steps and sub-steps may be flexible. For tablets, a formula task may define the steps and sub-steps to use. A custom recipe may be built, and/or lot tasks may be created. With the variety of steps, sub-steps, whole workflow may be customizable. Pharmacist can mix and match, e.g., for a brand new formulation. Some embodiments provide real-time capabilities, e.g., define how changes to inventory, orders, and formulas affect UIs, workflows. Some embodiments define what happens if a template changes while an order is being filled. Some embodiments obtain events from an inventory service and/or OMS. Suppose an OMS event occurs and there is no inventory. Suppose there is a need for 90 more units. Some embodiments may generate a task to make 90 and fulfill the order. Suppose a formula recipe changes, tasks that are being actively worked on may continue to use older version of template. New tasks may get new version of templates. Similar automation may be included for template changes. Some embodiments provide multi-user and/or parallel processing functionality. For example, multiple users may work on same formula, even if not concurrently. Steps may be sequential. For example, one person may need to finish a step/sub-step before handing it to a next person. For e.g., weigh, sample, clean lot tasks may be handled simultaneously.

In some embodiments, the receiving progressive inputs may be automated via a digital input device coupled to the workstation UI and wherein the digital input device is taken from the group consisting of a digital scale, a mixing device, and combinations thereof.

In some embodiments, digitally recording the receipt of pharmaceutical ingredients may include reading input from a bar code scanner indicating that the scanner is associated with the workstation UI.

In some embodiments, the method may include indicating in a formulation database that the active formulation supersedes all prior formulations.

The method may also include creating a compound pharmaceutical template via plurality of active formulations.

In some embodiments, rendering on a workstation UI identified by a computer readable indicia, instructions for formulating the compound pharmaceutical product, may be based on a ranking based on factors taken from the list consisting of a time since order was placed, time since prescription was approved, and inventory levels.

Thus, various techniques are described for automating compounding pharmacies.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A method of automating preparation of compound prescription at a plurality of workstations having integrated therein a user interface and a registered quantity input hardware, based on forecasted quantities, ordered quantities and simultaneous usage data, the method comprising:
obtaining a plurality of orders for compound prescriptions, via one or more web clients;
tracking information related to inventories including expiration dates for different lots of the inventories;
generating compounding tasks corresponding to the plurality of orders by analyzing ingredients necessary for fulfilling each order and assigning, for each order, a respective one or more lots for an inventory;
prioritizing and assigning the compounding tasks to a plurality of workstations, based on forecast demand, available supply, capacity, and the information related to the inventories;
providing one or more graphical user interfaces for a plurality of users, to process the plurality of orders at the plurality of workstations; and
in response to detecting a user of the plurality of users processing an order of the plurality of orders at a workstation of the plurality of workstations, updating the information related to the inventories, updating the compounding tasks, and/or providing an update on the one or more graphical user interfaces regarding a next compounding task performable at the workstation.

2. The method of claim 1, further comprising:
interfacing with at least one device selected from the group consisting of: one or more scales, one or more scanners, and one or more mixers; and
detecting a status of the order of the plurality of orders at the workstation, by obtaining one or more signals from the at least one device.

3. The method of claim 1, further comprising:
retrieving one or more templates corresponding to the plurality of orders, wherein each template includes a set of instructions for a workflow and a formula; and
providing, via the one or more graphical user interfaces, details to implement the formula using the workflow, at the plurality of workstations.

4. The method of claim 3, wherein the one or more templates are customizable based upon one or more devices connected to each workstation.

5. The method of claim 1, further comprising:
providing to a first set of users of the plurality of users a dashboard to update the one or more templates; and
upon receiving an update the one or more templates, providing the updates, via the one or more graphical user interfaces.

6. The method of claim 1, wherein the capacity corresponds to at least one of a number of workstations, and a number of users.

7. The method of claim 1, wherein the available supply is tracked based on tracking information related to the inventories, at one or more warehouses, and/or at the plurality of workstations.

8. The method of claim 1, wherein the forecast demand is obtained based on a forecasting model that is trained to predict demand based on historical data for compound prescription from the one or more web clients.

9. The method of claim 1, wherein prioritizing and assigning the compounding tasks to the plurality of workstations is based on planned shifts of the plurality of users, and/or operational efficiency of the plurality of workstations.

10. The method of claim 1, wherein prioritizing and assigning the compounding tasks to the plurality of workstations is based on forecasting, inventory, outstanding orders, a time an order was approved, and target days of coverage.

11. The method of claim 1, further comprising:
providing, via the one or more graphical user interfaces, for the user, while the user is processing the order, a real-time update regarding a weight of an ingredient and an expected range of weight for the ingredient according to a formula; and
in response to detecting the weight of the ingredient is stabilized, displaying a final weight of the ingredient, allowing the user to record, and/or automatically recording the final weight of the ingredient for the order.

12. The method of claim 1, further comprising:
providing, via the one or more graphical user interfaces, for a super user different from the user processing the order, after the user processes the order, a summary of the processing of the order; and
in response to receiving a negative input from the super user regarding an incorrect compounding for the order, requeuing a task corresponding to the order to the plurality of workstations.

13. The method of claim 1, wherein prioritizing and assigning the compounding tasks to the plurality of workstations is based on batching one or more tasks that use an ingredient.

14. The method of claim 1, wherein prioritizing and assigning the compounding tasks to the plurality of workstations is based on ordering tasks for one or more customers responsible for the plurality of orders received via the one or more web clients.

15. The method of claim 1, wherein prioritizing and assigning the compounding tasks to the plurality of workstations is based on a demand for one or more ingredients.

16. The method of claim 1, wherein prioritizing and assigning the compounding tasks to the plurality of workstations is based on task initiation prompted by a user request through the one or more graphical user interfaces thereby providing on-demand control.

17. The method of claim 1, wherein prioritizing and assigning the compounding tasks to the plurality of workstations is automatically triggered through a daemon to execute scheduled commands job.

18. The method of claim 1, wherein each compound includes a corresponding minimum inventory threshold to trigger a new order and/or a minimum batch size for a next order.

19. A computer system comprising:
one or more processors;
a display; and
memory;
wherein the memory stores one or more programs configured for execution by the one or more processors, and the one or more programs comprising instructions for:
obtaining a plurality of orders for compound prescriptions, via one or more web clients;
tracking information related to inventories including expiration dates for different lots of the inventories;
generating compounding tasks corresponding to the plurality of orders by analyzing ingredients necessary for fulfilling each order and assigning, for each order, a respective one or more lots for an inventory;
prioritizing and assigning the compounding tasks to a plurality of workstations, based on forecast demand, available supply, capacity, and the information related to the inventories;
providing one or more graphical user interfaces for a plurality of users, to process the plurality of orders at the plurality of workstations; and
in response to detecting a user of the plurality of users processing an order of the plurality of orders at a workstation of the plurality of workstations, updating the information related to the inventories, updating the compounding tasks, and/or providing an update on the one or more graphical user interfaces regarding a next compounding task performable at the workstation.

20. A non-transitory computer readable storage medium storing one or more programs configured for execution by a computer system having a display, one or more processors, and memory, the one or more programs comprising instructions for:
rendering on a pharmacist user interface (UI), a plurality of fields configured to receive input from a user having a first level authority;
receiving ingredient data to create an active formulation via a plurality of fields corresponding to i) pharmaceutical ingredients in a formulary; ii) quantities of the pharmaceutical ingredients making up a compound pharmaceutical product; and/or iii) instructions for combining the pharmaceutical ingredients to produce the compound pharmaceutical product;
receiving a digital order for the compound pharmaceutical product;
in response to receiving the digital order, rendering on a workstation UI identified by a computer readable indicia, instructions for formulating the compound pharmaceutical product and compounding fields configured to receive input from a user having a second level of authority that is different from the first level of authority;
digitally recording, based on the computer readable indicia, receipt of pharmaceutical ingredients for formulating the compound pharmaceutical product;
receiving progressive inputs based on authorization from the user having a second level of authority into the compounding fields, the inputs being associated with quantities of pharmaceutical ingredients that are dispensed proximate the workstation UI;
receiving a digital completion entry from at the workstation UI indicating the digital order has been created; and
reconciling the quantities of pharmaceutical ingredients that have been dispensed in the creation of the compound pharmaceutical product with indicated quantities in the instructions for formulating the compound pharmaceutical product.
